(19) United States
(12) Patent Application Publication
(10) Pub. No.: US 2020/0263231 A1
(43) Pub. Date: Aug. 20, 2020

(54) Fluorogenic dyes for high sensitivity DNA detection

(71) Applicant: LIFE TECHNOLOGIES CORPORATION, Carlsbad, CA (US)

(72) Inventors: Kyle GEE, Springfield, OR (US); Jongtae YANG, Eugene, OR (US)

(21) Appl. No.: 16/340,373

(22) PCT Filed: Nov. 1, 2017
(86) PCT No.: PCT/US2017/059594

§ 371 Date: Apr. 8, 2019

Related U.S. Application Data
(60) Provisional application No. 62/418,628, filed on Nov. 7, 2016.

(51) Int. Cl. C12Q 1/6806; C12Q 1/6818; C12Q 1/02; C09B 62/44; C09K 11/07; C07D 417/06

(52) U.S. Cl. CPC C12Q 1/6806; C12Q 1/6818; C12Q 1/02; C09B 62/4428; C09K 2211/1014; C07D 417/06; C09K 2211/1018; C09K 2211/1007; C09K 11/07

(57) ABSTRACT

The present disclosure is directed to unsymmetrical cyanine dyes comprising a substituted benzazolium ring system linked by a methine bridge to a quinolinium ring that contains a heteroatom. Compounds of formula (I) are provided herein. The compounds can be useful for fluorescent detection or quantification of nucleic acids. Related methods, uses, and kits are disclosed.

### CROSS REFERENCE

This application claims the benefit under 35 U.S.C. § 119(e) of U.S. Provisional Application No. 62/418,628, filed Nov. 7, 2016. The entire contents of the aforementioned applications are incorporated by reference herein.

### FIELD OF THE INVENTION

This disclosure relates to the field of compounds useful for fluorescent detection or quantification of nucleic acids.

### INTRODUCTION AND SUMMARY

In many fields it is useful or necessary to detect or quantify nucleic acids, e.g., in biological, biomedical, genetic, fermentation, aquaculture, agricultural, forensic and environmental research. Compounds that fluoresce when associated with nucleic acids have been useful to identify nucleic acids, qualitatively and quantitatively, in pure solutions and in biological samples. A fast, sensitive, and selective methodology that can detect minute amounts of nucleic acids in a variety of media, whether or not the nucleic acid is contained in cells is particularly desirable.

Disclosed herein are compounds that can provide improved sensitivity and/or selectivity for nucleic acids, such as double-stranded DNA (dsDNA) or single-stranded DNA (ssDNA) or other benefits, or at least provide the public with a useful choice.

In some embodiments, a compound of formula (I) is provided:

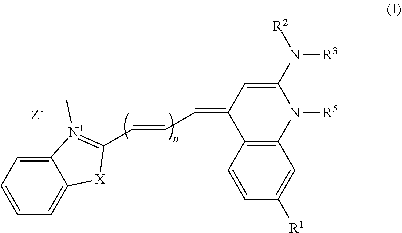

wherein:

Z⁻ is a biologically acceptable counterion; X is S, O, Se, or NR^{N1}, where R^{N1} is H or C₁₋₆ alkyl; n is 0, 1, or 2; R¹ is H or —O—C₁₋₆ alkyl; R² is C₂₋₆ alkyl; R³ is —(CH₂)ₘNRR′, or —(CH₂)ₘN⁺RR′R″, wherein:

m is 2-6, R and R′ are each independently a substituted or unsubstituted aryl or heteroaryl, or a substituted or unsubstituted C₁₋₆ alkyl, and R″ is H, a substituted or unsubstituted aryl or heteroaryl, or a substituted or unsubstituted C₁₋₆ alkyl; and R⁵ is an alkyl, alkenyl, polyalkenyl, alkynyl or polyalkynyl group having 1-6 carbons; a substituted or unsubstituted aryl or heteroaryl; or a substituted or unsubstituted cycloalkyl having 3-10 carbons;

wherein if R³ is —(CH₂)₃—N(CH₃)₂ or —(CH₂)₃—N⁺(CH₃)₃, then R² is ethyl or C₄₋₆ alkyl.

In some embodiments, R¹ is —O—C₁₋₄ alkyl. In some embodiments, R¹ is —O— methyl. In some embodiments, R² is C₁₋₄ alkyl. In some embodiments, R² is ethyl or C₄₋₆ alkyl. In some embodiments, R² is ethyl. In some embodiments, R² is n-propyl. In some embodiments, R² is n-butyl.

In some embodiments, R³ is —(CH₂)₃—N(CH₃)₂. In some embodiments, R³ is —(CH₂)ₘN⁺RR′R″.

In some embodiments, R is C₁₋₆ alkyl and R′ and R″ are each methyl. In some embodiments, R is ethyl. In some embodiments, R is n-propyl.

In some embodiments, a biologically acceptable counterion Zₐ⁻ is associated with R³. In some embodiments, Zₐ⁻ is a halide, sulfate, an alkanesulfonate, an arylsulfonate, phosphate, perchlorate, tetrafluoroborate, tetraarylboride, nitrate, or an anion of an aromatic or aliphatic carboxylic acid. In some embodiments, Zₐ⁻ is chloride, bromide, iodide, an alkanesulfonate, an arylsulfonate, or perchlorate. In some embodiments, Zₐ⁻ is bromide. In some embodiments, Zₐ⁻ is iodide. In some embodiments, Zₐ⁻ is chloride.

In some embodiments, R⁵ is a substituted or unsubstituted aryl or heteroaryl; or a substituted or unsubstituted cycloalkyl having 3-10 carbons. In some embodiments, R⁵ is a substituted or unsubstituted aryl or heteroaryl. In some embodiments, R⁵ is unsubstituted phenyl or phenyl substituted with 1, 2, or 3 instances of C₁₋₄ alkyl. In some embodiments, R⁵ is unsubstituted phenyl.

In some embodiments, Z⁻ is a halide, sulfate, an alkanesulfonate, an arylsulfonate, phosphate, perchlorate, tetrafluoroborate, tetraarylboride, nitrate, or an anion of an aromatic or aliphatic carboxylic acid. In some embodiments, Z⁻ is chloride, bromide, iodide, an alkanesulfonate, an arylsulfonate, or perchlorate. In some embodiments, Z⁻ is bromide. In some embodiments, Z⁻ is iodide. In some embodiments, Z⁻ is chloride.

In some embodiments, X is S.

In some embodiments, n is 0.

Also provided is a compound of formula (II):

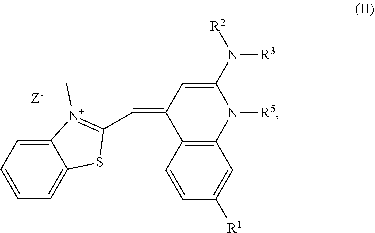

wherein Z⁻, R¹, R², R³, and R⁵ have values described herein.

Also provided is a compound of formula (III):

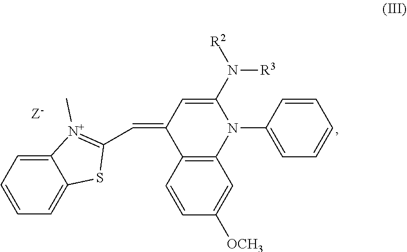

wherein Z⁻, R², and R³ have values described herein.

In some embodiments, R² is C₂₋₆ alkyl; and R³ is —(CH₂)ₘNRR′, or —(CH₂)ₘN⁺RR′R″, wherein m is 2-6, and R, R′ and R″ are each independently a substituted or unsubstituted aryl or heteroaryl, or a substituted or unsubstituted C₁₋₆ alkyl; wherein if R³ is —(CH₂)₃—N(CH₃)₂ or —(CH₂)₃—N⁺(CH₃)₃, then R² is ethyl or C₄₋₆ alkyl. In some embodiments, R² is ethyl or n-butyl. In some embodiments, R² is C₂₋₆ alkyl; R³ is —(CH₂)ₘN⁺RR′R″; R is C₁₋₄ alkyl which is unsubstituted or substituted with hydroxyl, or aryl which is unsubstituted or substituted with methyl, ethyl, or —O—CH₃; and R′ and R″ are each independently C₁₋₆ alkyl. In some embodiments, R² is ethyl, n-propyl, or n-butyl.

In some embodiments, R³ is —(CH₂)ₘN⁺RR′R″; R is ethyl or n-propyl; and R′ and R″ are each methyl. In some embodiments, R³ is —(CH₂)ₘN⁺RR′R″; R is —CH₂CH₂OH; and R′ and R″ are each methyl. In some embodiments, R³ is —(CH₂)ₘN⁺RR′R″; R is phenyl which is unsubstituted or substituted with methyl, ethyl, or —O—CH₃; and R′ and R″ are each methyl.

In some embodiments, R is phenyl or 3-methoxyphenyl.

In some embodiments the compound is

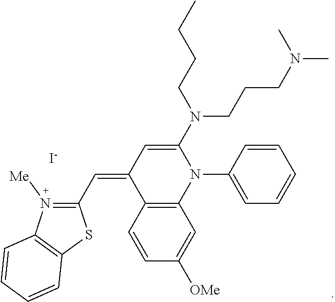

In some embodiments the compound is

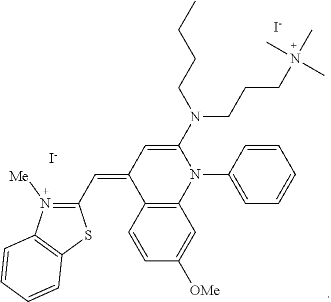

In some embodiments the compound is

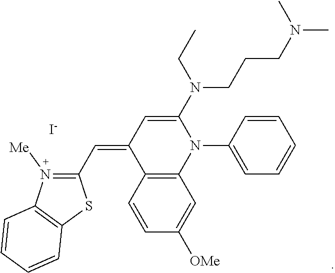

In some embodiments the compound is

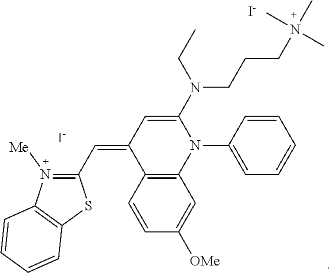

In some embodiments the compound is

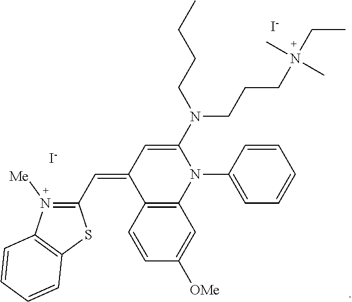

In some embodiments the compound is

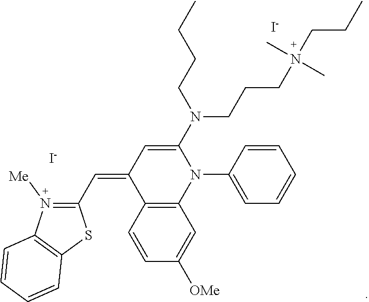

In some embodiments the compound is

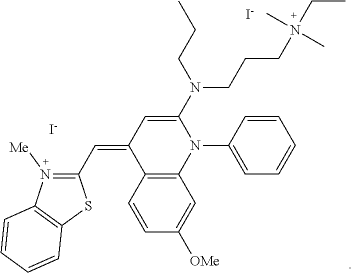

In some embodiments the compound is

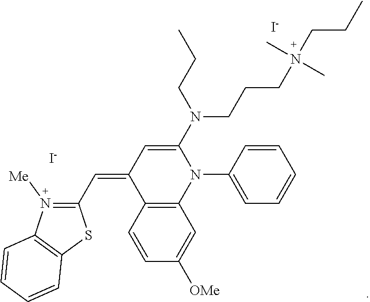

In some embodiments the compound is

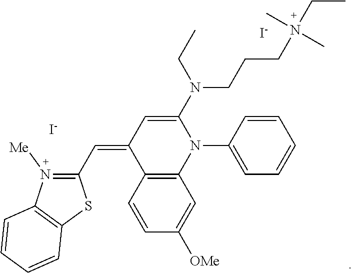

In some embodiments the compound is

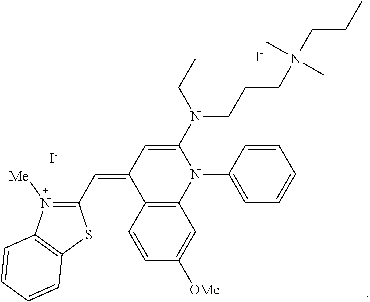

In some embodiments the compound is

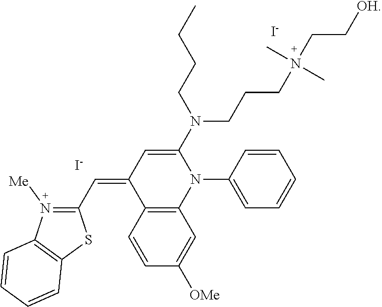

In some embodiments the compound is

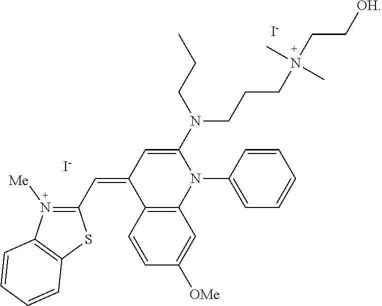

In some embodiments the compound is

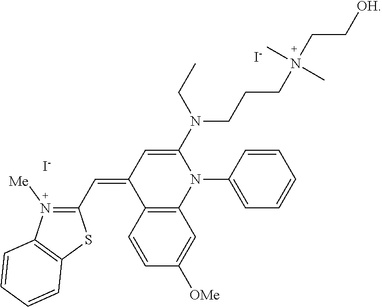

In some embodiments the compound is

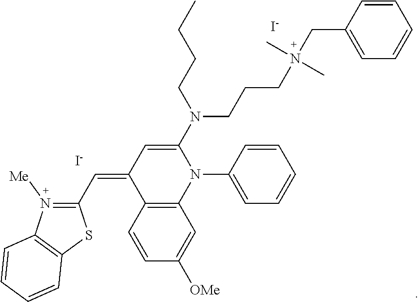

In some embodiments the compound is

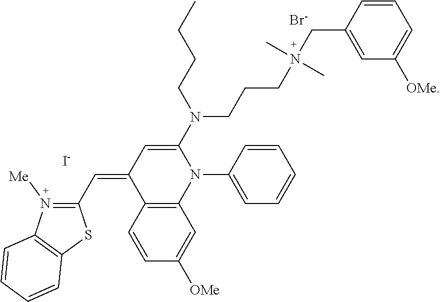

In some embodiments the compound is

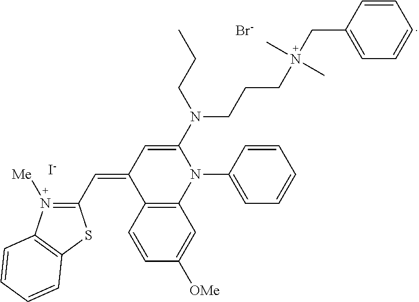

In some embodiments the compound is

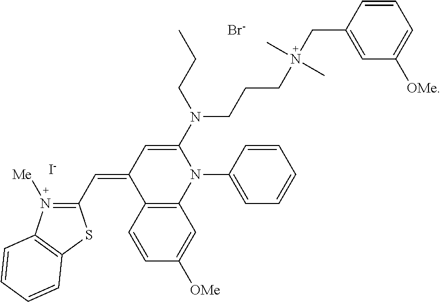

In some embodiments the compound is

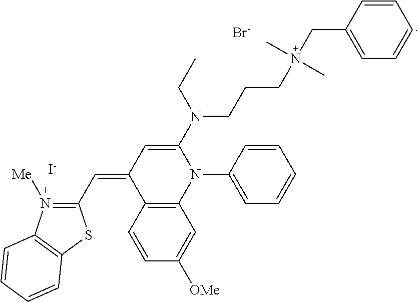

In some embodiments the compound is

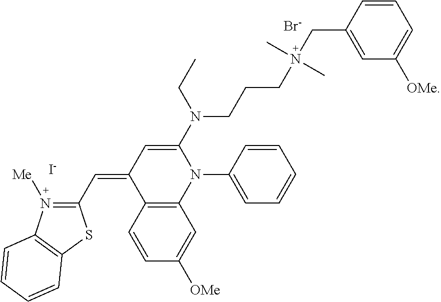

Also provided is a fluorescent complex comprising a compound disclosed herein non-covalently associated with a nucleic acid. Also provided is a method of staining a nucleic acid comprising contacting the nucleic acid with a compound disclosed herein. Also provided is a fluorescent complex formed by said method. Also provided is a method of labeling a nucleic acid comprising contacting the nucleic acid with a compound disclosed herein.

Also provided is a method of detecting a nucleic acid comprising exciting the fluorescent complex disclosed herein and detecting fluorescently emitted light.

Also provided is a method of detecting a nucleic acid in a sample, the method comprising: a) combining a compound disclosed herein with a sample that contains or is thought to contain a nucleic acid; b) incubating the sample and the compound for a sufficient amount of time for the compound to combine with the nucleic acid in the sample to form a compound-nucleic acid complex; c) illuminating the compound-nucleic acid complex with an appropriate wavelength to form an illuminated mixture; and d) detecting fluorescently emitted light thereby detecting the nucleic acid present in the illuminated mixture.

Also provided is a method of detecting a biological structure, the method comprising: a) combining a sample that contains or is thought to contain a specific biological structure with a compound disclosed herein; b) incubating the combined sample and compound for a time sufficient for the compound to combine with nucleic acids in the biological structure to form a pattern of compound-nucleic acid complexes having a detectable fluorescent signal that corresponds to the biological structure; and c) detecting the fluorescent signal that corresponds to the biological structure.

Also provided is a method of determining cell membrane integrity, the method comprising: a) incubating a sample containing one or more cells with a compound disclosed herein for a time sufficient for the compound to combine with intracellular nucleic acids to form an intracellular compound-nucleic acid complex having a detectable fluorescent signal; and b) determining cell membrane integrity of the one or more cells based on presence of the detectable fluorescent signal, where the presence of the detectable fluorescent signal indicates that the cell membrane integrity is compromised and the absence of the detectable fluorescent signal indicates that the cell membrane integrity is intact.

Also provided is a method of quantitating nucleic acids in a sample, the method comprising: a) combining a compound disclosed herein with a sample that contains or is thought to contain a nucleic acid; b) incubating the sample and the compound for a sufficient amount of time for the compound to combine with nucleic acid in the sample to form a compound-nucleic acid complex; c) illuminating the compound-nucleic acid complex with an appropriate wavelength to form an illuminated mixture; and d) quantifying the nucleic acid present in the illuminated mixture based on comparison of the detectable fluorescent signal in the illuminated mixture with a fluorescent standard characteristic of a given amount of a nucleic acid.

In some embodiments, a nucleic acid is dsDNA. In some embodiments, a nucleic acid is ssDNA. In some embodiments, a nucleic acid is RNA. In some embodiments, a nucleic acid is an RNA-DNA hybrid. In some embodiments, a nucleic acid has a length of about 8 to about 15 nucleotides, about 15 to about 30 nucleotides, about 30 to about 50 nucleotides, about 50 to about 200 nucleotides, about 200 to about 1000 nucleotides, about 1 kb to about 5 kb, about 5 kb to about 10 kb, about 10 kb to about 50 kb, about 50 kb to about 500 kb, about 500 kb to about 5 Mb, about 5 Mb to about 50 Mb, or about 50 Mb to about 500 Mb. In some embodiments, a nucleic acid is a plasmid, cosmid, PCR product, restriction fragment, or cDNA. In some embodiments, a nucleic acid is genomic DNA. In some embodiments, a nucleic acid is a natural or synthetic oligonucleotide. In some embodiments, a nucleic acid comprises modified nucleic acid bases or links. In some embodiments, a nucleic acid is in an electrophoresis gel. In some embodiments, a nucleic acid is in a cell. In some embodiments, a nucleic acid is in an organelle, virus, viroid, cytosol, cytoplasm, or biological fluid. In some embodiments, a nucleic acid is in or was obtained from a water sample, soil sample, foodstuff, fermentation process, or surface wash.

In some embodiments, exciting a fluorescent complex comprises exposing the fluorescent complex to light with a wavelength ranging from about 460 nm to about 520 nm, about 470 nm to about 510 nm, about 480 nm to about 510 nm, about 485 nm to about 505 nm, or about 490 nm to about 495 nm. In some embodiments, fluorescently emitted light is detected with a microscope, plate reader, fluorimeter, or photomultiplier tube. In some embodiments, the method further comprises quantifying the nucleic acid.

In some embodiments, a biological structure is a prokaryotic cell, a eukaryotic cell, a virus or a viroid. In some embodiments, a biological structure is a subcellular organelle that is intracellular or extracellular.

Also provided is a kit for detecting nucleic acid in a sample, wherein the kit comprises a compound disclosed herein and an organic solvent. In some embodiments, the kit further comprises instructions for detecting nucleic acid in a sample.

Also provided is a staining solution comprising a compound disclosed herein and a detergent or an organic solvent.

### BRIEF DESCRIPTION OF THE DRAWINGS

FIG. 1 shows structures of 6 compounds evaluated in selectivity experiments along with comparative compounds R1 and R2.

FIG. 2 shows fluorescent enhancement at different concentrations for selected compounds with dsDNA.

FIG. 3 shows fluorescent enhancement at different concentrations for selected compounds with ssDNA.

FIG. 4 shows dsDNA and ssDNA emissions for selected compounds at 500 ng/mL.

FIG. 5 shows fluorescent enhancement at different concentrations for selected compounds evaluated with double-stranded DNA (dsDNA).

FIG. 6 shows fluorescent enhancement at different concentrations for selected compounds evaluated with single-stranded DNA (ssDNA).

FIG. 7 shows dsDNA and ssDNA emissions for selected compounds at 500 ng/mL.

### DETAILED DESCRIPTION OF CERTAIN EMBODIMENTS

Reference will now be made in detail to certain embodiments of the disclosure, examples of which are illustrated in the accompanying drawings. While the disclosure will be described in conjunction with the illustrated embodiments, it will be understood that they are not intended to limit the disclosure to those embodiments. On the contrary, the disclosure is intended to cover all alternatives, modifications, and equivalents, which may be included within the disclosure as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a dye” includes a plurality of dyes and reference to “a cell” includes a plurality of cells and the like.

It will be appreciated that there is an implied “about” prior to the temperatures, concentrations, times, etc. discussed in the present disclosure, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “kit” refers to a packaged set of related components, such as one or more compounds or compositions and one or more related materials such as solvents, solutions, buffers, instructions, or desiccants.

“Substituted” as used herein refers to a molecule wherein one or more hydrogen atoms are replaced with one or more non-hydrogen atoms, functional groups or moieties. By example, an unsubstituted nitrogen is —NH₂, while a substituted nitrogen is —NHCH₃. Exemplary substituents include but are not limited to halogen, e.g., fluorine and chlorine, (C₁-C₈) alkyl, sulfate, sulfonate, sulfone, amino, ammonium, amido, nitrile, nitro, lower alkoxy, phenoxy, aromatic, phenyl, polycyclic aromatic, heterocycle, water-solubilizing group, linkage, and linking moiety. In some embodiments, substituents include, but are not limited to,

—X, —R, —OH, —OR, —SR, —SH, —NH₂, —NHR, —NR₂, —⁺NR₃, —N═NR₂, —CX₃, —CN, —OCN, —SCN, —NCO, —NCS, —NO, —NO₂, —N₂⁺, —N₃, —NHC(O)R, —C(O)R, —C(O)NR₂, —S(O)₂O⁻, —S(O)₂R, —OS(O)₂OR, —S(O)₂NR, —S(O)R, —OP(O)(OR)₂, —P(O)(OR)₂, —P(O)(O⁻)₂, —P(O)(OH)₂, —C(O)R, —C(O)X, —C(S)R, —C(O)OR, —CO₂⁻, —C(S)OR, —C(O)SR, —C(S)SR, —C(O)NR₂, —C(S)NR₂, —C(NR)NR₂, where each X is independently a halogen and each R is independently —H, C₁-C₆ alkyl, C₅-C₁₄ aryl, heterocycle, or linking group.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined herein, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the definitions provided herein are not intended to include impermissible substitution patterns (e.g., methyl substituted with 5 fluoro groups). Such impermissible substitution patterns are well known to the skilled artisan.

The compounds disclosed herein may exist in unsolvated forms as well as solvated forms, including hydrated forms. These compounds may exist in multiple crystalline or amorphous forms. In general, all physical forms are equivalent for the uses described herein and are intended to be within the scope of the present disclosure. The compounds disclosed herein may possess asymmetric carbon atoms (i.e., chiral centers) or double bonds; the racemates, diastereomers, geometric isomers and individual isomers of the compounds described herein are within the scope of the present disclosure. The compounds described herein may be prepared as a single isomer or as a mixture of isomers.

Where substituent groups are specified by their conventional chemical formulae and are written from left to right, they equally encompass the chemically identical substituents, which would result from writing the structure from right to left, e.g., —CH₂O— is intended to also recite —OCH₂—.

It will be understood that the chemical structures that are used to define the compounds disclosed herein are each representations of one of the possible resonance structures by which each given structure can be represented. Further, it will be understood that by definition, resonance structures are merely a graphical representation used by those of skill in the art to represent electron delocalization, and that the present disclosure is not limited in any way by showing one particular resonance structure for any given structure.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

“Alkyl” means a saturated or unsaturated, branched, straight-chain, or cyclic hydrocarbon radical derived by the removal of one hydrogen atom from a single carbon atom of a parent alkane, alkene, or alkyne. Typical alkyl groups consist of 1 to 12 saturated and/or unsaturated carbons, including, but not limited to, methyl, ethyl, propyl, butyl, and the like. In some embodiments, an alkyl is a monovalent saturated aliphatic hydrocarbyl group having from 1 to 10 carbon atoms, e.g., 1 to 6 carbon atoms, e.g. 1, 2, 3, 4, 5 or 6 carbon atoms. “Alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, e.g., 1 to 3, or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxylalkyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, substituted sulfonyl, sulfonyloxy, thioacyl, thiol, alkylthio, and substituted alkylthio, wherein said substituents are defined herein. Particular substituted alkyl groups comprise a reactive group for direct or indirect linking to a carrier molecule or solid support, for example, but not limited to, alkyl substituted by carboxyl or a carboxyl ester (e.g. an activated ester such as an N-hydroxysuccinimide ester) and alkyl substituted by aminocarbonyl —CONHR where R is an organic moiety as defined below with reference to the term “aminocarbonyl”, e.g. a C₁-C₁₀ (e.g. C₁-C₆) alkyl terminally substituted by a reactive group (Rₓ) including, but not limited to, carboxyl, carboxylester, maleimide, succinimidyl ester (SE), sulfodichlorophenyl (SDP) ester, sulfotetrafluorophenyl (STP) ester, tetrafluorophenyl (TFP) ester, pentafluorophenyl (PFP) ester, nitrilotriacetic acid (NTA), aminodextran, and cyclooctyne-amine.

“Alkylsulfonate” is —(CH₂)ₙ—SO₃H, wherein n is an integer from 1 to 6.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy. In some embodiments, an alkoxy is —OR where R is (C₁-C₆) alkyl.

“Substituted alkoxy” refers to the group —O-(substituted alkyl), wherein substituted alkyl is defined herein.

“Alkyldiyl” means a saturated or unsaturated, branched, straight chain or cyclic hydrocarbon radical of 1 to 20 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl, 1,3-propyldiyl, 1,4-butyldiyl, and the like.

“Aryl” or “Ar” means a monovalent aromatic hydrocarbon radical of 6 to 20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like. In some embodiments, an aryl is a monovalent aromatic carboxylic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, e.g., 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, substituted sulfonyl, sulfonyloxy, thioacyl, thiol, alkylthio, and substituted alkylthio, wherein said substituents are defined herein.

“Aryleno” means an aromatic ring fused at two contiguous aryl carbons of a compound, i.e. a divalent bridge radical having two adjacent monovalent radical centers derived by the removal of one hydrogen atom from each of two adjacent carbon atoms of a parent aromatic ring system. Attaching an aryleno bridge radical, e.g. benzeno, to a parent aromatic ring system results in a fused aromatic ring system, e.g. naphthalene. Typical aryleno groups include, but are not limited to: [1,2]benzeno, [1,2]naphthaleno and [2,3]naphthaleno.

“Aryldiyl” means an unsaturated cyclic or polycyclic hydrocarbon radical of 6-20 carbon atoms having a conjugated resonance electron system and at least two monovalent radical centers derived by the removal of two hydrogen atoms from two different carbon atoms of a parent aryl compound.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, e.g., 1 to 3, or 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Alkenyl” refers to alkenyl groups having from 2 to 6 carbon atoms, e.g., 2 to 4 carbon atoms, and having at least 1, e.g., from 1 to 2 sites of alkenyl unsaturation. Such groups are exemplified, for example, by vinyl, allyl, but-3-en-1-yl, and propenyl.

“Substituted alkenyl” refers to alkenyl groups having from 1 to 3 substituents, e.g., 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic, wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂— alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂— heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NRC(O)NR′R″ where R is hydrogen or alkyl and R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NRC(S)NR′R″ where R is hydrogen or alkyl and R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NRSO₂NR′R″ where R is hydrogen or alkyl and R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkyenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkyenyl, aryl, substituted aryl, heteroaryl, substituted heteroaryl, heterocyclic and substituted heterocyclic are as defined herein.

“Amidino” refers to the group —C(═NR′″)R′R″ where R′, R″, and R′″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R′ and R″ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aryloxy” refers to the group —O-aryl, where aryl is as defined herein, that includes, by way of example, phenoxy and naphthoxy.

“Substituted aryloxy” refers to the group —O-(substituted aryl), where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkynyl” refers to alkynyl groups having from 2 to 6 carbon atoms and e.g., 2 to 3 carbon atoms and having at least 1, e.g., from 1 to 2 sites of alkynyl unsaturation.

“Substituted alkynyl” refers to alkynyl groups having from 1 to 3 substituents, e.g., 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Carbonyl” refers to the divalent group —C(O)— which is equivalent to —C(═O)—.

“Carboxyl” or “carboxy” refers to —COOH or salts thereof.

“Carboxyl alkyl” or “carboxyalkyl” refers to the group —(CH₂)ₙCOOH, where n is 1-6.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O— substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O— cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic, wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O— substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation, e.g., from 1 to 2 sites of >C═C< ring unsaturation.

“Substituted cycloalkyl” and “substituted cycloalkenyl” refer to a cycloalkyl or cycloalkenyl group having from 1 to 5, e.g., 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“H” indicates hydrogen.

“Halo” or “halogen” refers to fluoro, chloro, bromo and iodo.

“Hydroxy” or “hydroxyl” refers to the group —OH.

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” means any ring system having at least one non-carbon atom in a ring, e.g. nitrogen, oxygen, and sulfur. In some embodiments, a heterocycle is a saturated or unsaturated group having a single ring or multiple condensed rings, including fused bridged and spiro ring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties. Heterocycles include, but are not limited to: pyrrole, indole, furan, benzofuran, thiophene, benzothiophene, 2-pyridyl, 3-pyridyl, 4-pyridyl, 2-quinolyl, 3-quinolyl, 4-quinolyl, 2-imidazole, 4-imidazole, 3-pyrazole, 4-pyrazole, pyridazine, pyrimidine, pyrazine, cinnoline, pthalazine, quinazoline, quinoxaline, 3-(1,2,4-N)-triazolyl, 5-(1,2,4-N)-triazolyl, 5-tetrazolyl, 4-(1-O, 3-N)-oxazole, 5-(1-O, 3-N)-oxazole, 4-(1-S, 3-N)-thiazole, 5-(1-S, 3-N)-thiazole, 2-benzoxazole, 2-benzothiazole, 4-(1,2,3-N)-benzotriazole, and benzimidazole.

“Substituted heterocyclic” or “substituted heterocycloalkyl” or “substituted heterocyclyl” refers to heterocyclyl groups that are substituted with from 1 to 5, e.g., 1 to 3 of the same substituents as defined for substituted cycloalkyl.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

“Hydrazinyl” refers to the group —NHNH₂— or ═NNH—.

“Substituted hydrazinyl” refers to a hydrazinyl group, wherein a non-hydrogen atom, such as an alkyl group, is appended to one or both of the hydrazinyl amine groups. An example of substituted hydrazinyl is —N(alkyl)-NH₂ or ═N⁺(alkyl)-NH₂.

“Nitro” refers to the group —NO₂.

“Oxo” refers to the atom (═O) or (—O—).

“Spirocyclyl” refers to divalent saturated cyclic group from 3 to 10 carbon atoms having a cycloalkyl or heterocyclyl ring with a spiro union (the union formed by a single atom which is the only common member of the rings) as exemplified by the following structure:

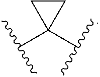 [SMILES: *C1(*)CC1]

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂— substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl), wherein substituted alkyl is as defined herein.

The term “detectable response” as used herein refers to an occurrence of or a change in, a signal that is directly or indirectly detectable either by observation or by instrumentation. In some embodiments, the detectable response is an optical response resulting in a change in the wavelength distribution patterns or intensity of absorbance or fluorescence or a change in light scatter, fluorescence lifetime, fluorescence polarization, or a combination of the above parameters.

The term “dye” as used herein refers to a compound that emits light to produce an observable detectable signal.

As used herein, the term “fluorophore” or “fluorogenic” refers to a compound or a composition that demonstrates a change in fluorescence upon binding to a biological compound or analyte of interest and/or upon cleavage by an enzyme. The fluorophores of the present disclosure may be substituted to alter the solubility, spectral properties or physical properties of the fluorophore.

As used herein, “a pharmaceutically acceptable salt” or “a biologically compatible salt” is a counterion that is not toxic as used, and does not have a substantially deleterious effect on biomolecules. Examples of such salts include, among others, K⁺, Na⁺, Cs⁺, Li⁺, Ca²⁺, Mg²⁺, Cl⁻. AcO⁻, and alkylammonium or alkoxyammonium salts.

The term “linker” or “L”, as used herein, refers to a single covalent bond or a moiety comprising series of stable covalent bonds, the moiety often incorporating 1-40 plural valent atoms selected from the group consisting of C, N, O, S and P that covalently attach the fluorogenic or fluorescent compounds to another moiety such as a chemically reactive group or a biological and non-biological component. The number of plural valent atoms in a linker may be, for example, 0, 1, 2, 3, 4, 5, 6, 7, 8, 9, 10, 20, 25, 30 or a larger number up to 40 or more. A linker may be linear or non-linear; some linkers have pendant side chains or pendant functional groups, or both. Examples of such pendant moieties are hydrophilicity modifiers, for example solubilizing groups like, e.g. sulfo (—SO₃H or —SO₃—). In certain embodiments, L is composed of any combination of single, double, triple or aromatic carbon-carbon bonds, carbon-nitrogen bonds, nitrogen-nitrogen bonds, carbon-oxygen bonds and carbon-sulfur bonds. Exemplary linking members include a moiety that includes —C(O)NH—, —C(O)O—, —NH—, —S—, —O—, and the like. Linkers may, by way of example, consist of a combination of moieties selected from alkyl; —C(O)NH—; —C(O)O—; —NH—; —S—; —O—; —C(O)—; —S(O)ₙ— where n is 0, 1 or 2; —O—; 5- or 6-membered monocyclic rings; and optional pendant functional groups, for example sulfo, hydroxy and carboxy. The moiety formed by a linker bonded to a reactive group (Rₓ) may be designated -L-Rₓ. The reactive group may be reacted with a substance reactive therewith, whereby the linker becomes bonded to a conjugated substance (S_{c}) and may be designated -L-S_{c}, or in some cases, the linker may contain a residue of a reactive group (e.g. the carbonyl group of an ester) and may be designated “-L_{R}”. A “cleavable linker” is a linker that has one or more cleavable groups that may be broken by the result of a reaction or condition. The term “cleavable group” refers to a moiety that allows for release of a portion, e.g., a fluorogenic or fluorescent moiety, of a conjugate from the remainder of the conjugate by cleaving a bond linking the released moiety to the remainder of the conjugate. Such cleavage is either chemical in nature, or enzymatically mediated. Exemplary enzymatically cleavable groups include natural amino acids or peptide sequences that end with a natural amino acid.

The term “solid support,” as used herein, refers to a matrix or medium that is substantially insoluble in liquid phases and capable of binding a molecule or particle of interest. Solid supports suitable for use herein include semi-solid supports and are not limited to a specific type of support. Useful solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as SEPHAROSE® (GE Healthcare), poly(acrylate), polystyrene, poly(acrylamide), polyol, agarose, agar, cellulose, dextran, starch, FICOLL® (GE Healthcare), heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

As used herein, the term “staining” is a technique used in microscopy to enhance contrast in the microscopic image. Stains and dyes are frequently used to highlight structures in biological tissues and cells. Staining also involves adding a dye to a substrate to quantify or qualify the presence of a specific compound, such as a protein, nucleic acid, lipid or carbohydrate. Biological staining is also used to mark cells in flow cytometry and to flag proteins or nucleic acids in gel electrophoresis. Staining is not limited to biological materials and can be used to study the morphology of other materials such as semi-crystalline polymers and block copolymers.

As used herein, “about” refers to a value that is 10% more or less than or equal to a stated value, gives results functionally equivalent to the stated value, or rounds to the stated value.

“Or” is used in the inclusive sense, i.e., equivalent to “and/or,” unless the context requires otherwise.

Compound 1, shown below, is sometimes used as a comparative compound:

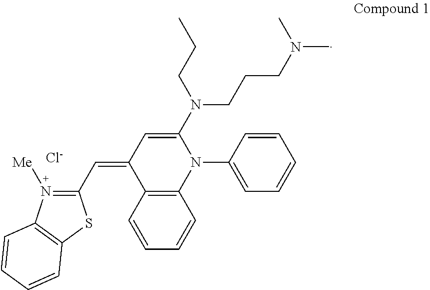

### Exemplary Compounds

Provided herein are substituted unsymmetrical cyanine dyes, kits and compositions including such dyes, as well as methods using such dyes for detecting and quantifying nucleic acids. In some embodiments, the disclosed dyes and compositions are used advantageously for the detection and quantification of ssDNA and dsDNA. It was surprisingly found that certain dye compounds and methods disclosed herein can be used to detect ssDNA and dsDNA in vitro and in cells with ever increasing sensitivity. Certain dye compounds disclosed herein are unsymmetrical cyanine dyes that have a combination of specific structural variations on the quinolinium ring that unexpectedly afforded the molecules with substantially increased fluorescence, both increased absolute fluorescence and increased signal:noise (S/N) fluorescence, when bound to ssDNA and dsDNA. In addition, this structural combination afforded increased permeability of live cells. Other structural variations either afforded no benefit or even a decrement in DNA sensing ability. Accordingly, certain compounds, compositions and methods provided herein can overcome many of the disadvantages associated with conventional nucleic acid binding dyes and/or provide new opportunities for the use and quantification of ssDNA and dsDNA.

In some embodiments, a compound provided herein comprises: 1) a first heterocyclic ring system that is a substituted benzazolium ring; 2) a bridging methine; and 3) a second heterocyclic ring system that is a quinolinium ring system, wherein at position 7 is a —O—C₁₋₆ alkyl group, and at position 2 is —NR²R³, wherein R² is C₂₋₆ alkyl and R³ is —(CH₂)ₘNRR′, or —(CH₂)ₘN⁺RR′R″, wherein m is 2-6, R and R′ are each independently a substituted or unsubstituted aryl or heteroaryl, or a substituted or unsubstituted C₁₋₆ alkyl, and R″ is H, a substituted or unsubstituted aryl or heteroaryl, or a substituted or unsubstituted C₁₋₆ alkyl. As used herein, the numbering of the positions of the quinolinium ring is as follows:

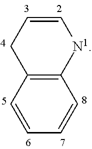

Disclosed are compounds 2-65 shown below:

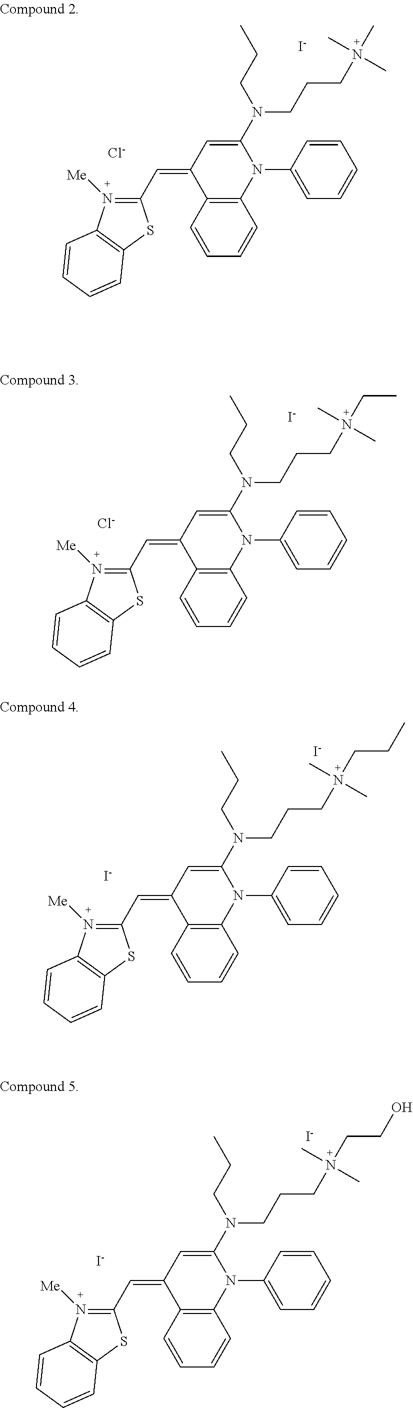

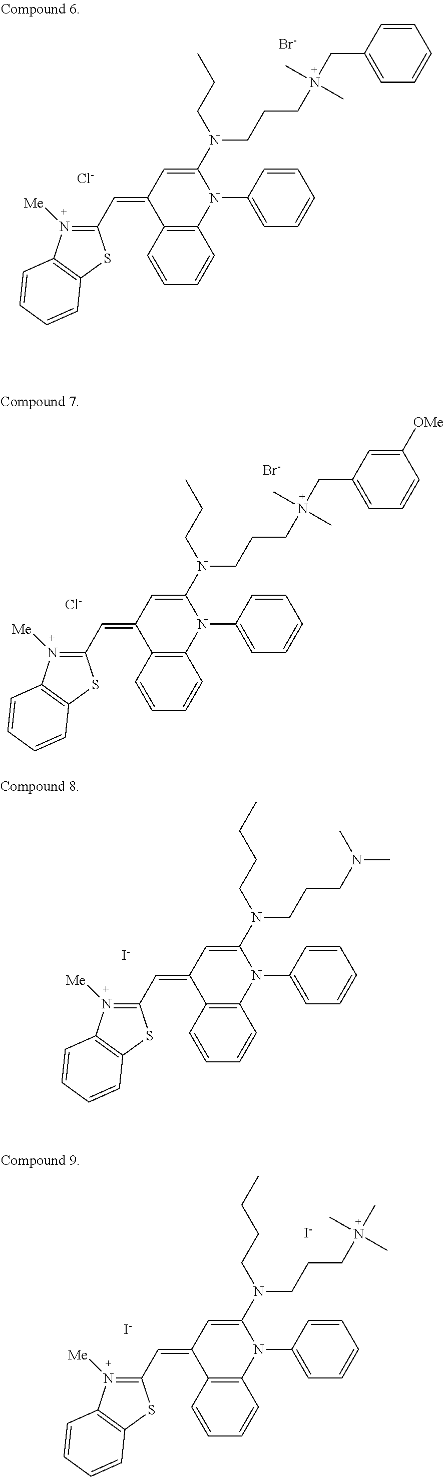

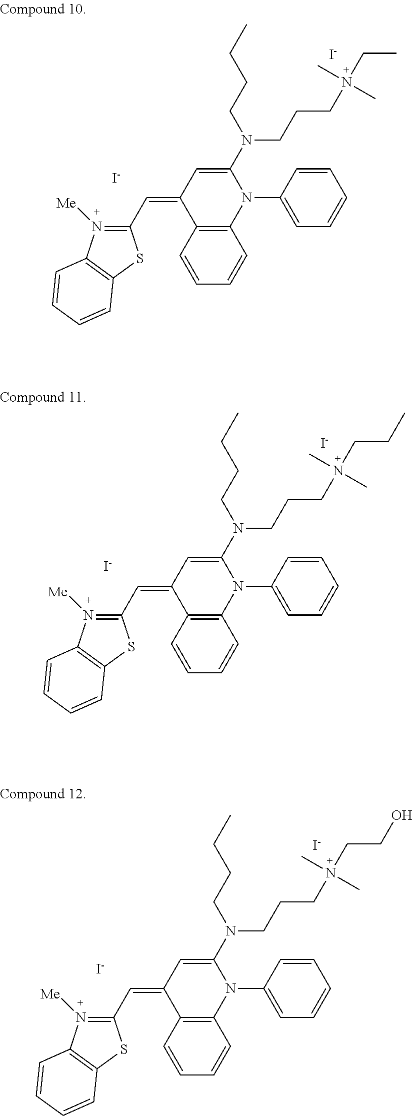

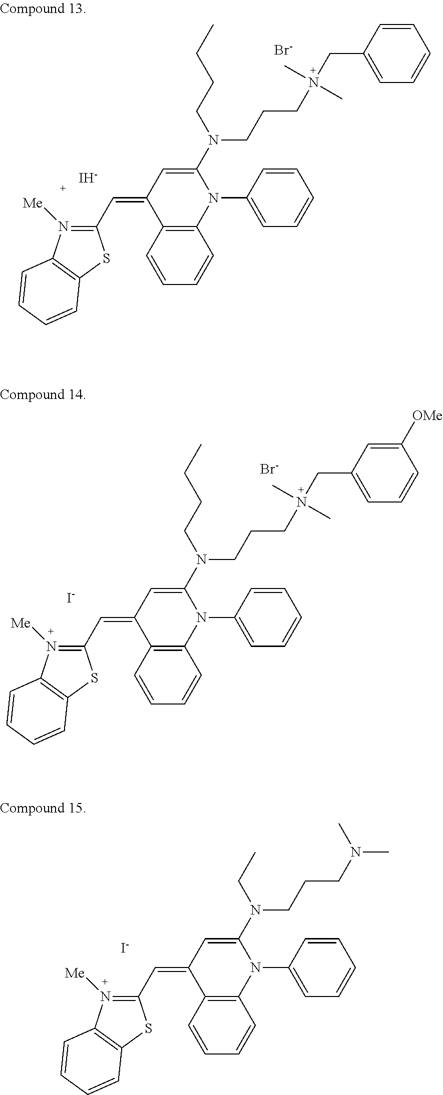

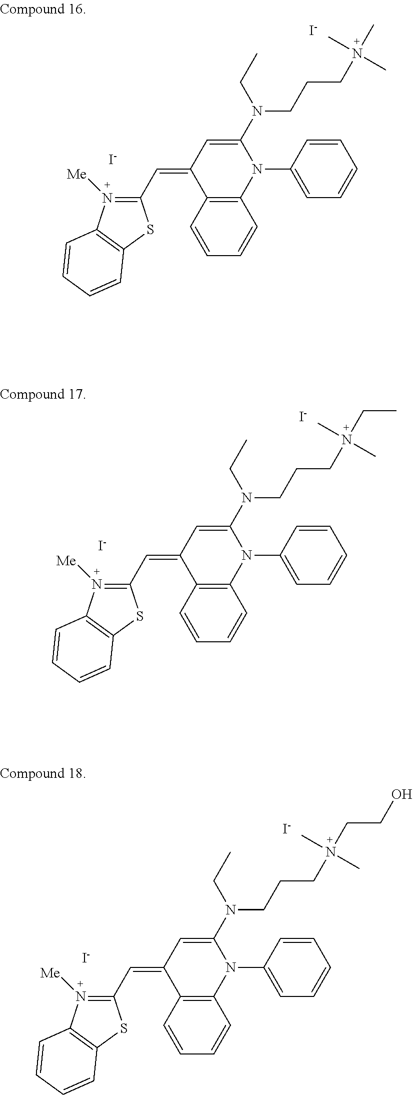

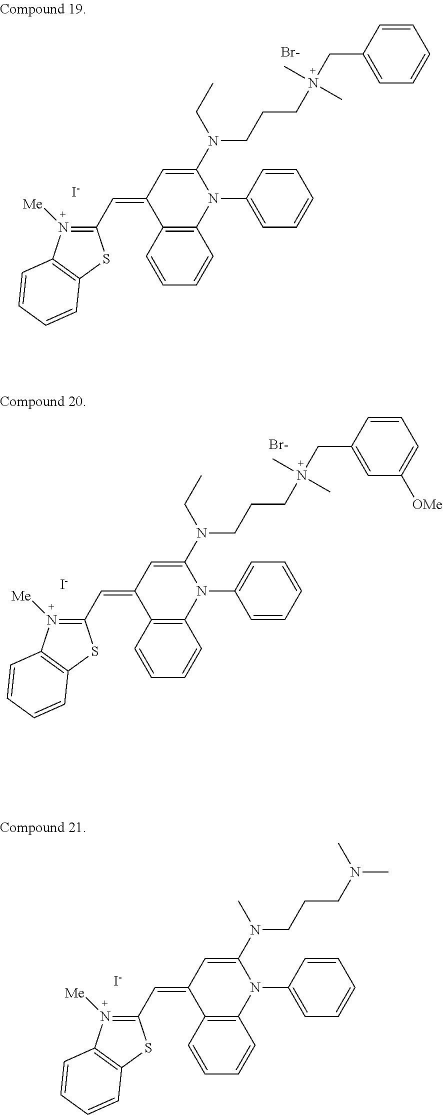

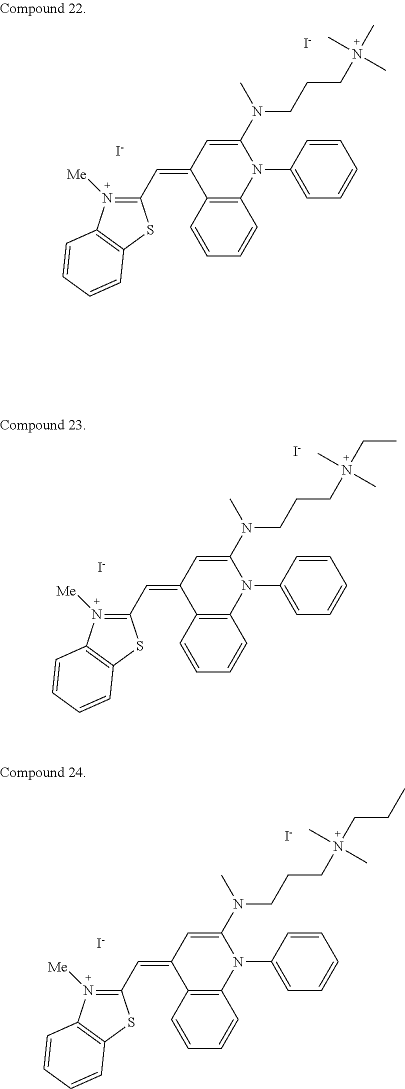

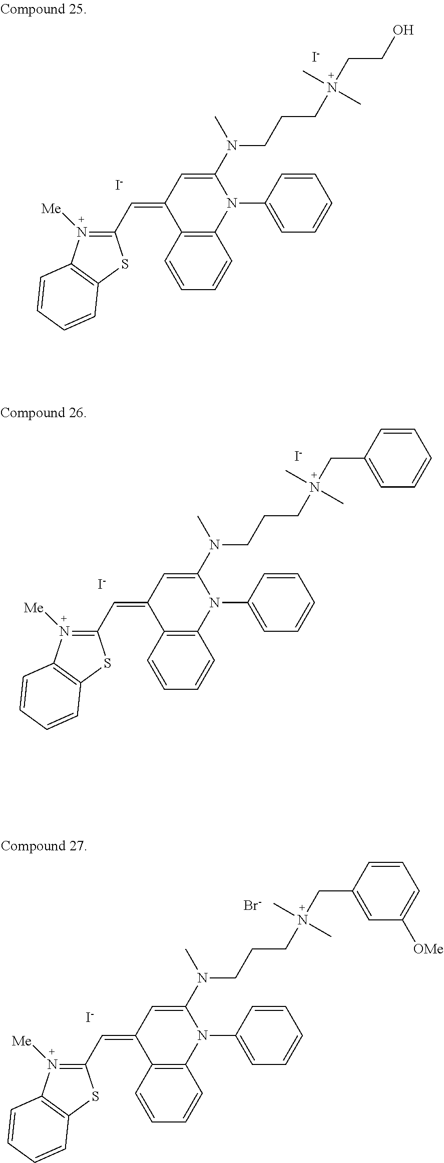

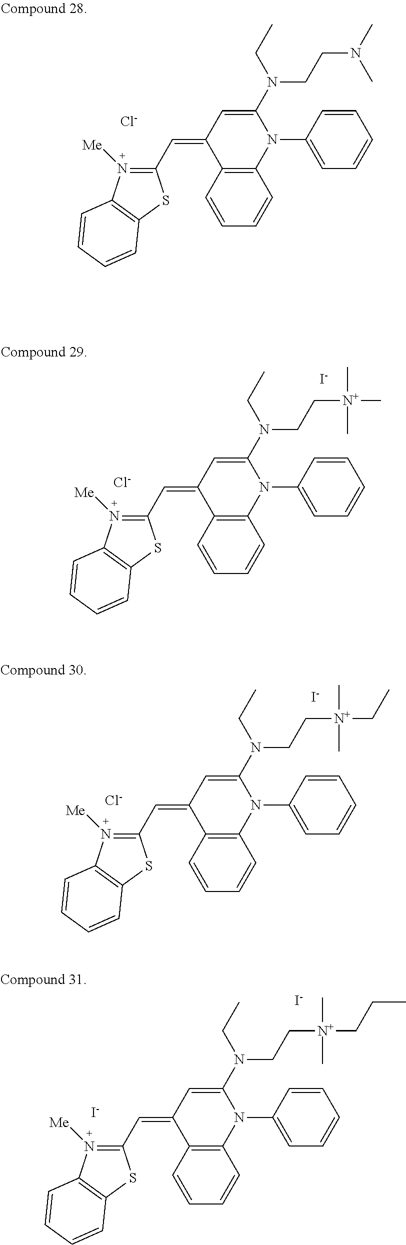

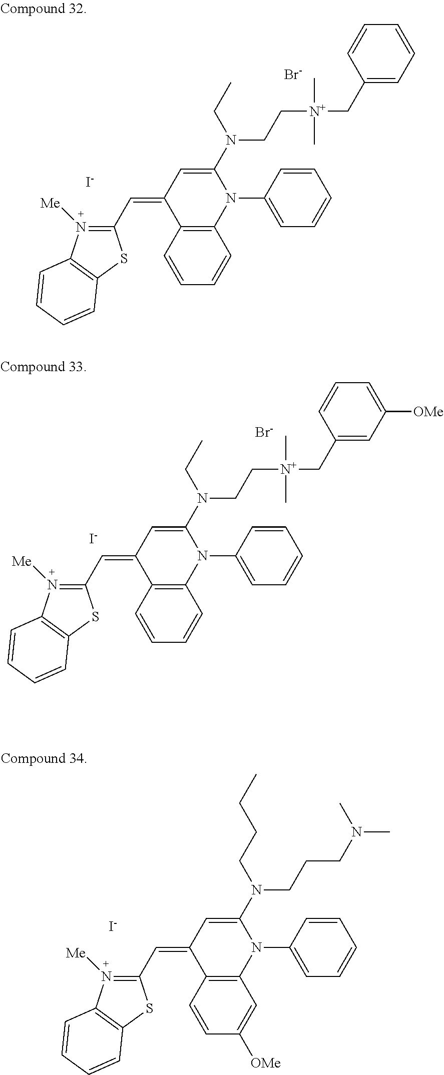

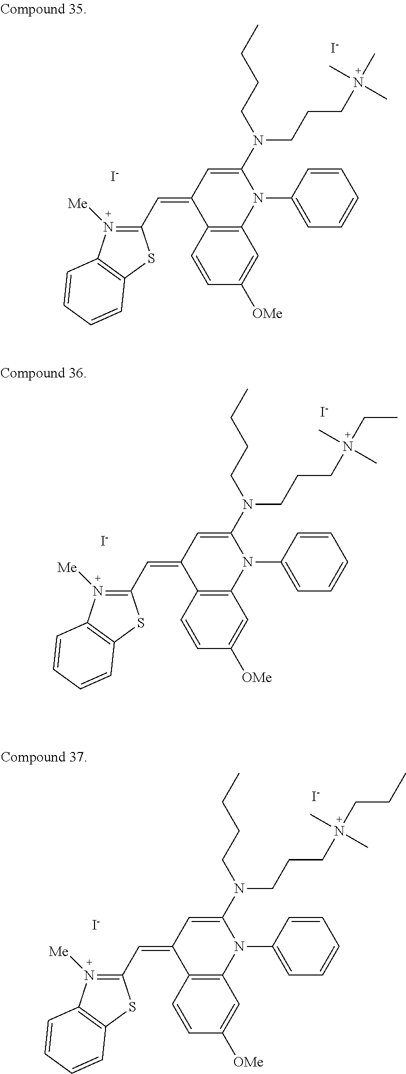

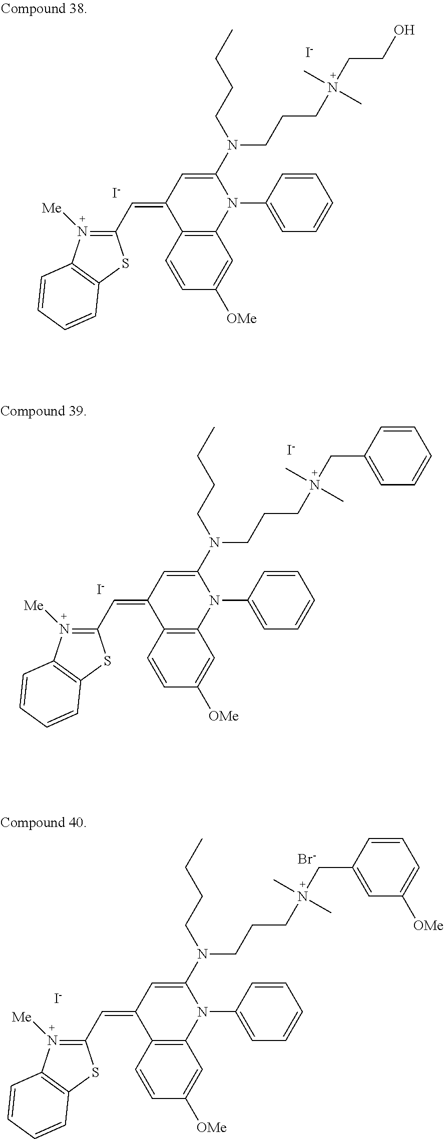

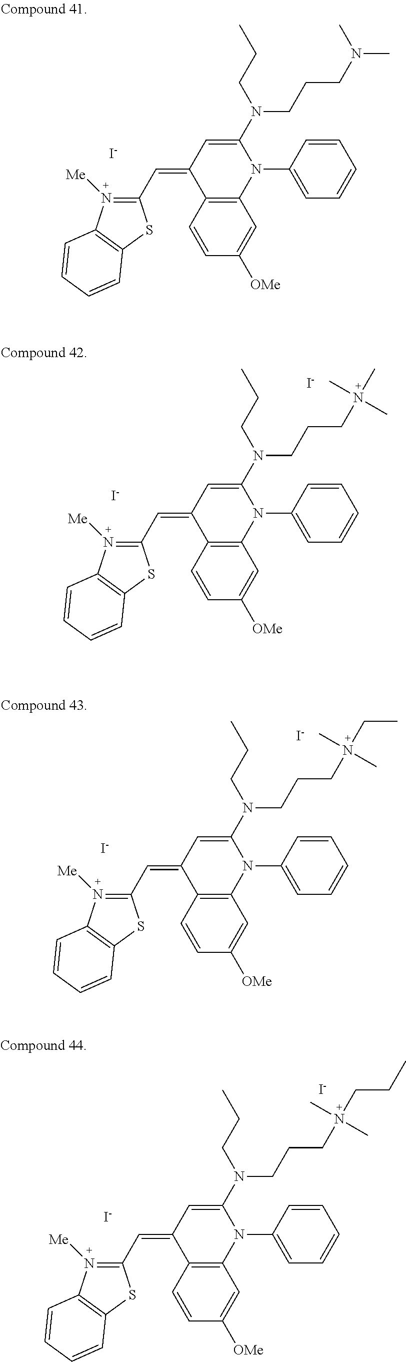

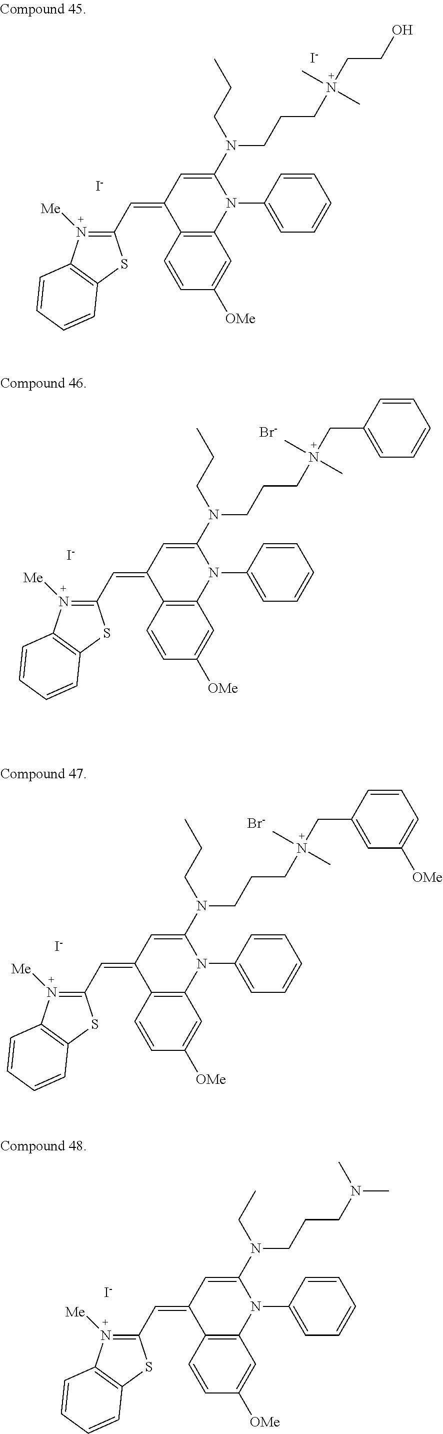

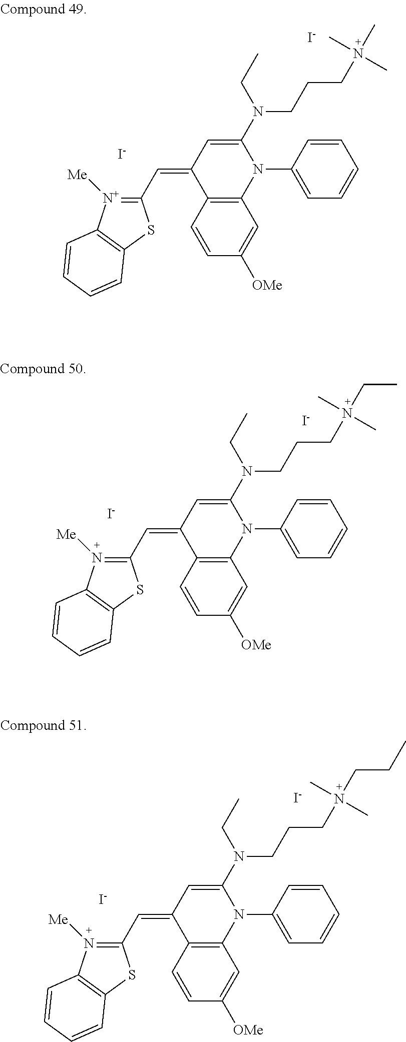

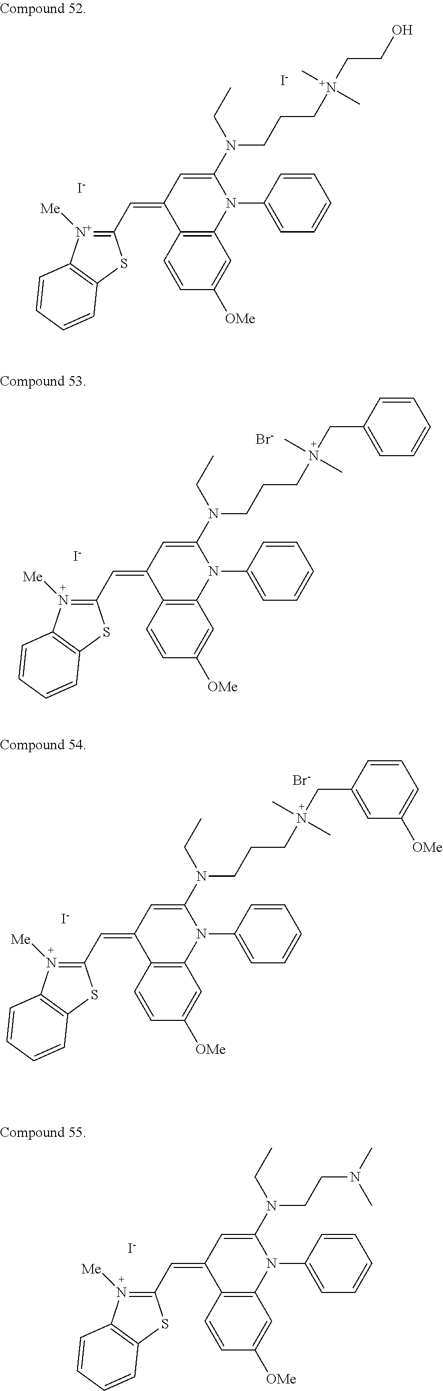

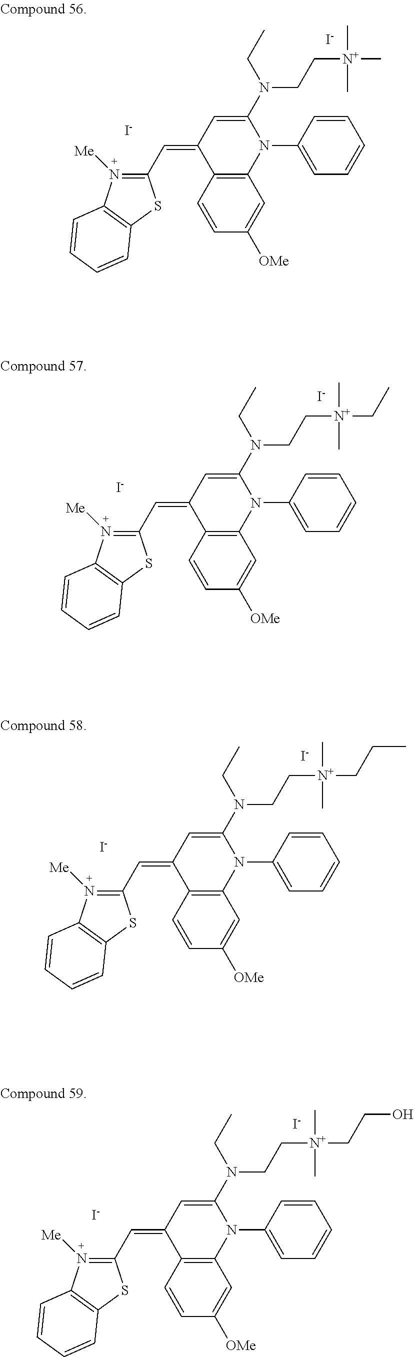

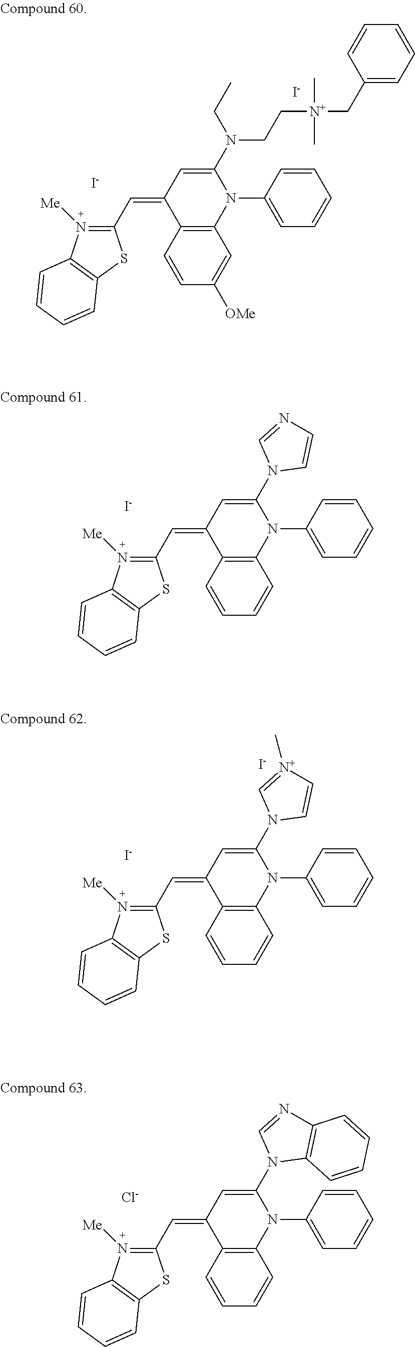

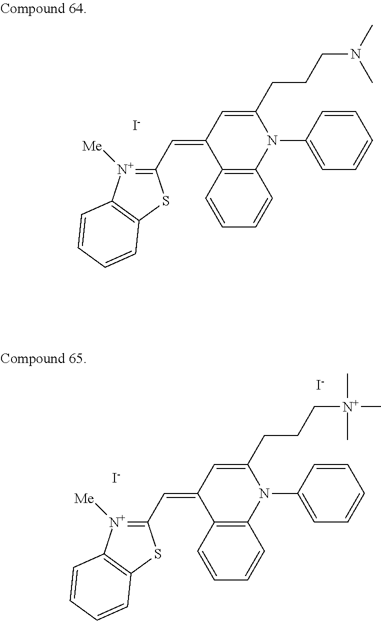 [SMILES: CN(C)CCCC1=C/C(=C/c2sc3ccccc3[n+]2C)c2ccccc2N1c1ccccc1 ; [I-] ; C[n+]1c(/C=C2/C=C(CCC[N+](C)(C)C)N(c3ccccc3)c3ccccc32)sc2ccccc21 ; [I-] ; [I-]]

In some embodiments, the disclosure relates to a compound selected from compounds 2-65 shown above.

Compounds of formula (I) are disclosed:

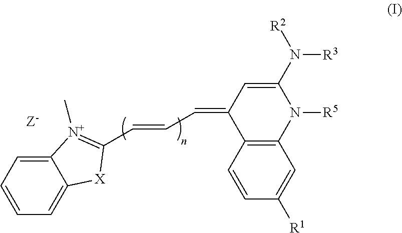

wherein Z is a biologically acceptable counterion; X is S, O, Se, or NR^{N1}, where R^{N1} is H or C₁₋₆ alkyl; n is 0, 1, or 2; R¹ is H or —O—C₁₋₆ alkyl; R² is C₂₋₆ alkyl; R³ is —(CH₂)ₘNRR′, or —(CH₂)ₘN⁺RR′R″, m is 2-6, R and R′ are each independently a substituted or unsubstituted aryl or heteroaryl, or a substituted or unsubstituted C₁₋₆ alkyl, and R″ is H, a substituted or unsubstituted aryl or heteroaryl, or a substituted or unsubstituted C₁₋₆ alkyl; and R⁵ is an alkyl, alkenyl, polyalkenyl, alkynyl or polyalkynyl group having 1-6 carbons; a substituted or unsubstituted aryl or heteroaryl; or a substituted or unsubstituted cycloalkyl having 3-10 carbons; wherein if R³ is —(CH₂)₃—N(CH₃)₂ or —(CH₂)₃—N⁺(CH₃)₃, then R² is ethyl or C₄₋₆ alkyl.

In some embodiments, a compound disclosed herein is a compound of formula (II):

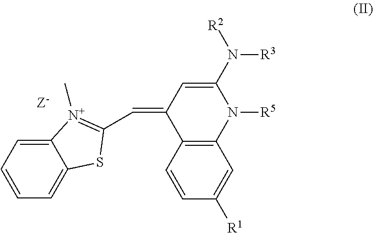

wherein Z⁻ is a biologically acceptable counterion; R¹ is H or —O—C₁₋₆ alkyl; R² is C₂₋₆ alkyl; R³ is —(CH₂)ₘNRR′, or —(CH₂)ₘN⁺RR′R″, m is 2-6, R and R′ are each independently a substituted or unsubstituted aryl or heteroaryl, or a substituted or unsubstituted C₁₋₆ alkyl, and R″ is H, a substituted or unsubstituted aryl or heteroaryl, or a substituted or unsubstituted C₁₋₆ alkyl; and R⁵ is an alkyl, alkenyl, polyalkenyl, alkynyl or polyalkynyl group having 1-6 carbons; a substituted or unsubstituted aryl or heteroaryl; or a substituted or unsubstituted cycloalkyl having 3-10 carbons; wherein if R³ is —(CH₂)₃—N(CH₃)₂ or —(CH₂)₃—N⁺(CH₃)₃, then R² is ethyl or C₄₋₆ alkyl.

In some embodiments, a compound disclosed herein is a compound of formula (III):

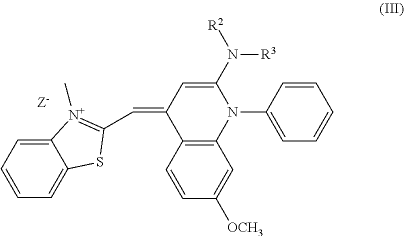

wherein Z⁻ is a biologically acceptable counterion; R² is C₂₋₆ alkyl; R³ is —(CH₂)ₘNRR′, or —(CH₂)ₘN⁺RR′R″, m is 2-6, R and R′ are each independently a substituted or unsubstituted aryl or heteroaryl, or a substituted or unsubstituted C₁₋₆ alkyl, and R″ is H, a substituted or unsubstituted aryl or heteroaryl, or a substituted or unsubstituted C₁₋₆ alkyl; wherein if R³ is —(CH₂)₃—N(CH₃)₂ or —(CH₂)₃—N⁺(CH₃)₃, then R² is ethyl or C₄₋₆ alkyl.

The following embodiments relating to Z⁻, X, n, R¹, R², R³, and R⁵ or parts or counterions thereof are described with respect to each and every formula above to which they can apply.

In some embodiments, R¹ is —O—C₁₋₄ alkyl, e.g., —O-methyl, —O-ethyl, —O-n-propyl, or —O-isopropyl.

In some embodiments, R² is C₁₋₄ alkyl, e.g., methyl, ethyl, n-propyl, isopropyl, n-butyl, isobutyl, or t-butyl. In some embodiments, R² is not n-propyl. In some embodiments, R² is not n-propyl or isopropyl.

In some embodiments, R³ is —(CH₂)ₘN⁺RR′R″. In some embodiments, R R′ and R″ are each methyl, or R′ is methyl and R″ is H. In some embodiments, R is C₁₋₆ alkyl, e.g., methyl, ethyl, n-propyl, isopropyl, n-butyl, isobutyl, or t-butyl. In some embodiments, R is not methyl. In some embodiments, R is ethyl or n-propyl. In some embodiments, R is —CH₂CH₂OH. In some embodiments, R is phenyl which is unsubstituted or substituted with methyl, ethyl, or —O—CH₃. In some embodiments, R is C₁₋₄ alkyl which is unsubstituted or substituted with hydroxyl, or aryl which is unsubstituted or substituted with methyl, ethyl, or —O—CH₃. In some embodiments, R is phenyl. In some embodiments, R is 3-methoxyphenyl.

R³ can be associated with a biologically acceptable counterion Zₐ⁻, e.g., a halide, sulfate, an alkanesulfonate, an arylsulfonate, phosphate, perchlorate, tetrafluoroborate, tetraarylboride, nitrate, or an anion of an aromatic or aliphatic carboxylic acid. In some embodiments, Zₐ⁻ is chloride, bromide, iodide, an alkanesulfonate, an arylsulfonate, or perchlorate.

In some embodiments, R⁵ is a substituted or unsubstituted aryl or heteroaryl. In some embodiments, R⁵ is substituted or unsubstituted phenyl, naphthyl, pyridinyl, pyrrolyl, indolyl, thiophenyl, or furanyl. In some embodiments, R⁵ is a substituted or unsubstituted cycloalkyl having 3, 4, 5, 6, 7, 8, 9, or 10 carbons. In some embodiments, R⁵ is substituted with 1-3 methyls. In some embodiments, R⁵ is unsubstituted.

In some embodiments, Z⁻ is a halide, sulfate, an alkanesulfonate, an arylsulfonate such as phenylsulfonate, phosphate, perchlorate, tetrafluoroborate, tetraarylboride such as tetraphenylboride, nitrate, or an anion of an aromatic or aliphatic carboxylic acid, such as acetate or benzylate.

The selected compounds listed as compounds 1-65 above are not intended to be an exclusive list of the dyes of the present disclosure. Numerous modifications, substitutions, and alterations in substituents and compound structure are possible without departing from the spirit and scope of the disclosure.

### Exemplary Compositions, Complexes, Methods, Uses, and Kits

In general, the compounds disclosed herein are minimally fluorescent, if at all, in aqueous solution but are fluorescent when in a complex with a nucleic acid. A nucleic acid can be stained by contacting the nucleic acid with a compound disclosed herein. A fluorescent complex can be formed by contacting a nucleic acid with a compound disclosed herein. The nucleic acid can be DNA, e.g., dsDNA or ssDNA. The nucleic acid can also be RNA or an RNA-DNA hybrid. The compounds can be used to label or detect nucleic acids in a wide variety of samples, such as in aqueous solutions, electrophoretic gels, and a wide variety of cells, including microorganisms.

A compound can be combined with a sample that contains or is thought to contain a nucleic acid polymer, and then the mixture of compound and sample is incubated for a time sufficient for the compound to combine with nucleic acid polymers in the sample to form one or more compound-nucleic acid complexes having a detectable fluorescent signal. The characteristics of the compound-nucleic acid complex, including the presence, location, intensity, excitation and emission spectra, fluorescence polarization, fluorescence lifetime, and other physical properties of the fluorescent signal can be used to detect, differentiate, sort, quantitate, and/or analyze aspects or portions of the sample. The compounds of the disclosure are optionally used in conjunction with one or more additional reagents (e.g., detectably different fluorescent reagents), including compounds of the same class having different spectral properties.

Staining Solution.

In some embodiments, the subject compound is prepared for use by dissolving the compound in a staining solution, e.g., an aqueous or aqueous-miscible solution that is compatible with the sample and the intended use. For biological samples, where minimal perturbation of cell morphology or physiology is desired, the staining solution is selected accordingly. For solution assays, the staining solution in some embodiments does not perturb the native conformation of the nucleic acid undergoing evaluation. At pH higher than 8 and lower than 6.5, fluorescence of the compound-nucleic acid complex and stability of the compounds is reduced. High concentrations of organic solvents, cations, and oxidizing agents also generally reduce fluorescence, as does the ionic detergent sodium dodecyl sulfate (SDS) at concentrations ≥0.01%. A number of staining solution additives, however, do not interfere with the fluorescence of the compound-nucleic acid complex (e.g. urea up to 8M; CsCl up to 1 g/mL; formamide up to 50% of the solution; and sucrose up to 40%). The compounds generally have greater stability in buffered solutions than in water alone; and agents that reduce the levels of free oxygen radicals, such as j-mercaptoethanol, contribute to the stability of the compounds.

A staining solution can be made by dissolving the compound directly in an aqueous solvent such as water, a buffer solution, such as buffered saline (in some embodiments non-phosphate for some viability discrimination applications), a Tris(hydroxymethyl)aminomethane (TRIS) buffer (e.g., containing EDTA), or a water-miscible organic solvent such as dimethylsulfoxide (DMSO), dimethylformamide (DMF), or a lower alcohol such as methanol or ethanol. The compound is usually preliminarily dissolved in an organic solvent (in some embodiments 100% DMSO) at a concentration of greater than or equal to about 100-times that used in the staining solution, then diluted one or more times with an aqueous solvent such as water or buffer, such that the compound is present in an effective amount.

An effective amount of compound is the amount sufficient to give a detectable fluorescence response in combination with nucleic acids. The compound concentration in the solution must be sufficient both to contact the nucleic acids in the sample and to combine with the nucleic acids in an amount sufficient to give a signal, but too much compound will cause problems with background fluorescence. In some embodiments staining solutions for cellular samples have a compound concentration greater than or equal to 0.1 nM and less than or equal to 50 μM, such as greater than or equal to 1 nM and less than or equal to 10 μM, e.g., between 0.5 and 5 μM. In general, lower concentrations of compounds are required for eukaryotes than for prokaryotes, and for compounds with higher sensitivity. Staining solutions for electrophoretic gels can have a compound concentration of greater than or equal to 0.1 μM and less than or equal to 10 μM, such as about 0.5-2 μM; the same holds true where the compound is added to the gel (pre-cast) before being combined with nucleic acids. Staining solutions for detection and quantitation of free nucleic acids in solution can have a concentration of 0.1 μM-2 μM. The optimal concentration and composition of the staining solution is determined by the nature of the sample (including physical, biological, biochemical and physiological properties), the nature of the compound-sample interaction (including the transport rate of the compound to the site of the nucleic acids), and the nature of the analysis being performed, and can be determined according to standard procedures such as those described in examples below.

Sample Types.

The compound is combined with a sample that contains or is thought to contain a nucleic acid. The nucleic acid in the sample may be RNA or DNA, or a mixture or a hybrid thereof. Any DNA is optionally single-, double-, triple-, or quadruple-stranded DNA; any RNA is optionally single stranded (“ss”) or double stranded (“ds”). The nucleic acid may be a natural polymer (biological in origin) or a synthetic polymer (modified or prepared artificially). The nucleic acid polymer (e.g. containing at least 8 bases or base pairs) may be present as nucleic acid fragments, oligonucleotides, or larger nucleic acid polymers with secondary or tertiary structure. The nucleic acid is optionally present in a condensed phase, such as a chromosome. The nucleic acid polymer optionally contains one or more modified bases or links or contains labels that are non-covalently or covalently attached. For example, the modified base can be a naturally occurring modified base such as Ψ (pseudouridine) in tRNA, 5-methylcytosine, 6-methylaminopurine, 6-dimethylaminopurine, 1-methylguanine, 2-methylamino-6-hydroxypurine, 2-dimethylamino-6-hydroxypurine, or other known minor bases (see, e.g. Davidson, THE BIOCHEMISTRY OF THE NUCLEIC ACIDS (1976)) or is synthetically altered to contain an unusual linker such as morpholine derivatized phosphates (AntiVirals, Inc., Corvallis, Oreg.), or peptide nucleic acids such as N-(2-aminoethyl)glycine units (Wittung, et al., Nature 368, 561 (1994)) or contain a simple reactive functional group (<10 carbons) that is an aliphatic amine, carboxylic acid, alcohol, thiol or hydrazine, or contain a fluorescent label or other hapten, such as inosine, bromodeoxyuridine, iododeoxyuridine, biotin, digoxigenin, 2,4-dinitrophenyl, where the label is originally attached on the nucleotide or on the 3′ or 5′ end of the polymer, or ligands non-covalently attached to the nucleic acids. The sensitivity of the compounds for polymers containing primarily modified bases and links may be diminished by interference with the binding mode. Some embodiments of the compounds inhibit non-specific nuclease activity but not restriction endonuclease activity at certain compound:base pair ratios.

The sample that contains the nucleic acid is optionally a biological structure (i.e. an organism or a discrete unit of an organism), or a solution (including solutions that contain biological structures), or a solid or semi-solid material. Consequently, the nucleic acid used to practice the disclosure is optionally free in solution, immobilized in or on a solid or semi-solid material, extracted from a biological structure (e.g. from lysed cells, tissues, organisms or organelles), or remains enclosed within a biological structure. The nucleic acid can also be found in the cytosol of a cell, cytoplasm of a cell, or the nucleic acid can be extracellular. In order for the nucleic acids to bind to the compounds, it is necessary that the nucleic acids be in an aqueous environment to contact the compound, even if the nucleic acids are not enclosed in a biological structure.

The sample nucleic acid can be natural or synthetic and can be obtained from a wide variety of sources. The presence of the nucleic acid in the sample may be due to natural biological processes, or the result of a successful or unsuccessful synthesis or experimental methodology, undesirable contamination, or a disease state. The nucleic acid may be endogenous to the natural source or introduced as foreign material, such as by infection, transfection, or therapeutic treatment. Nucleic acids may be present in all, or only part, of a sample, and the presence of nucleic acids may be used to distinguish between individual samples, or to differentiate a portion or region within a single sample, or to identify the sample or characteristics of the sample.

In some embodiments, the sample containing nucleic acids is a cell or is an aqueous or aqueous-miscible solution that is obtained directly from a liquid source or as a wash from a solid material (organic or inorganic) or a growth medium in which cells have been introduced for culturing or a buffer solution in which nucleic acids or biological structures have been placed for evaluation. Where the nucleic acids are in cells, the cells are optionally single cells, including microorganisms, or multiple cells associated with other cells in two or three dimensional layers, including multicellular organisms, embryos, tissues, biopsies, filaments, biofilms, etc. Alternatively, the sample is a solid, optionally a smear or scrape or a retentate removed from a liquid or vapor by filtration. In one aspect of the disclosure, the sample is obtained from a biological fluid, including separated or unfiltered biological fluids such as urine, cerebrospinal fluid, blood, lymph fluids, tissue homogenate, interstitial fluid, cell extracts, mucus, saliva, sputum, stool, physiological secretions or other similar fluids. Alternatively, the sample is obtained from an environmental source such as soil, water, or air; or from an industrial source such as taken from a waste stream, a water source, a supply line, or a production lot. Industrial sources also include fermentation media, such as from a biological reactor or food fermentation process such as brewing; or foodstuffs, such as meat, gain, produce, or dairy products.

Where the nucleic acid is present in a solution, the sample solution can vary from one of purified or synthetic nucleic acids such as oligonucleotides to crude mixtures such as cell extracts or homogenates or other biological fluids, or dilute solutions from biological, industrial, or environmental sources. In some cases, it is desirable to separate the nucleic acids from a mixture of biomolecules or fluids in the solution prior to combination with the compound. Numerous techniques exist for separation and purification of nucleic acids from generally crude mixtures with other proteins or other biological molecules. These include such means as chromatographic techniques and electrophoretic techniques, using a variety of supports or solutions or in a flowing stream. Alternatively, mixtures of nucleic acids may be treated with RNase or DNase so that the polymer that is not degraded in the presence of the nuclease can be discriminated from degradation products using the subject compounds.

The source and type of sample, as well as the use of the compound, will determine which compound characteristics, and thus which compounds, will be most useful for staining a particular sample. For most applications, compounds are selected to give a quantum yield greater than or equal to about 0.3, e.g., greater than or equal to 0.6. when bound to nucleic acid; in some embodiments the compounds have a quantum yield ≤0.01 when not bound to nucleic acid, and a fluorescence enhancement greater than or equal to about 200 fold, e.g., greater than or equal to 1000 fold. Where the fluorescence of the compound-nucleic acid complex is detected utilizing sustained high intensity illumination (e.g. microscopy), compounds with rate of photobleaching lower than commonly used compounds (e.g. fluorescein) are preferred, particularly for use in live cells. The relatively low toxicity of the compounds to living systems generally enables the examination of nucleic acids in living samples with little or no perturbation caused by the compound itself. Where the compound must penetrate cell membranes or a gel, more permeant compounds are preferred, although some cells readily take up compounds that are shown to be impermeant to other cells by means other than passive diffusion across cell-membranes, e.g. by phagocytosis or other types of ingestion. Compounds that rapidly and readily penetrate cells do not necessarily rapidly penetrate gels. In applications where the nucleic acids are stained on a gel, the compound is also selected to have a high binding affinity (e.g., K_{d}≥10⁻⁶ M); whereas in applications where the nucleic acid will be prestained prior to undergoing a separation step, such as gel or capillary electrophoresis, even higher binding affinity (e.g., K_{d}≥10⁻⁸ M) is preferred to ensure good separation. In staining nucleic acids in solution, high binding affinity translates into greater sensitivity to small amounts of nucleic acid, but compounds with a moderate binding affinity (e.g., 10⁻⁶ M≤K_{d}≤10⁻⁸ M) are more effective over a greater dynamic range. The photostability, toxicity, binding affinity, quantum yield, and fluorescence enhancement of compounds are determined according to standard methods known in the art.

Formation of Compound-Nucleic Acid Complex.

The sample is combined with the staining solution by any means that facilitates contact between the compound and the nucleic acid. In some embodiments, the contact occurs through simple mixing, as in the case where the sample is a solution. A staining solution containing the compound may be added to the nucleic acid solution directly or may contact the nucleic acid solution in a liquid separation medium such as an electrophoretic liquid or matrix, sieving matrix or running buffer, or in a sedimentation (e.g. sucrose) or buoyant density gradient (e.g. containing CsCl), or on an inert matrix such as a blot or gel, a testing strip, or any other solid or semi-solid support. Suitable supports also include, but are not limited to, polymeric microparticles (including paramagnetic microparticles), polyacrylamide and agarose gels, nitrocellulose filters, computer chips (such as silicon chips for photolithography), natural and synthetic membranes, liposomes and alginate hydrogels, and glass (including optical filters), and other silica-based and plastic support. The compound is optionally combined with the nucleic acid solution prior to undergoing gel or capillary electrophoresis or micro-capillary electrophoresis, gradient centrifugation, or other separation step, during separation, or after the nucleic acids undergo separation. Alternatively, the compound is combined with an inert matrix or solution in a capillary prior to addition of the nucleic acid solution, as in pre-cast gels, capillary electrophoresis, micro-capillary electrophoresis, or preformed density or sedimentation gradients.

Where the nucleic acids are enclosed in a biological structure, the sample can be incubated with the compound. While permeant compounds of this class have shown an ability to permeate biological structures rapidly and completely upon addition of the compound solution, any other technique that is suitable for transporting the compound into the biological structure is also a valid method of combining the sample with the subject compound. Some cells actively transport the compounds across cell membranes (e.g. endocytosis or ingestion by an organism or other uptake mechanism) regardless of their cell membrane permeability. Suitable artificial means for transporting the compounds (or preformed compound-nucleic acid complexes) across cell membranes include, but are not limited to, action of chemical agents such as detergents, enzymes or adenosine triphosphate; receptor- or transport protein-mediated uptake; liposomes or alginate hydrogels; phagocytosis; pore-forming proteins; microinjection; electroporation; hypoosmotic shock; or minimal physical disruption such as scrape loading, patch clamp methods, or bombardment with solid particles coated with or in the presence of the compounds. In some embodiments, where intact structures are desired, the methods for staining cause minimal disruption of the viability of the cell and integrity of cell or intercellular membranes. Alternatively, the cells are fixed and treated with routine histochemical or cytochemical procedures, particularly where pathogenic organisms are suspected to be present. The cells can be fixed immediately after staining with an aldehyde fixative that keeps the compound in the cells. In some cases, live or dead cells may even be fixed prior to staining without substantially increasing cell membrane permeability of previously live cells so that only cells that were already dead prior to fixation stain with the cell-impermeant compound.

The sample is combined with the compound for a time sufficient to form the fluorescent nucleic acid-compound complex, in some embodiments the minimum time required to give a high signal-to-background ratio. Although all of the novel class of compounds are nucleic acid binding dyes, detectable fluorescence within biological structures or in gels requires entry of the compound across the biological membrane or into gels. Optimal staining with a particular compound is dependent upon the physical and chemical nature of the individual sample and the sample medium, as well as the property being assessed. The optimal time is usually the minimum time required for the compound, in the concentration being used, to achieve the highest target-specific signal while avoiding degradation of the sample over time and minimizing all other fluorescent signals due to the compound. For example, where the compound is chosen to be selective for a particular nucleic acid polymer or type of cell, the optimal time is usually the minimum time required to achieve the highest signal on that polymer or type of cell, with little to no signal from other nucleic acids or other cell types. Over time, undesirable staining may occur as even very low rates of diffusion transport small amounts of the very sensitive compounds into other cell types, or as the cell membranes degrade, or as nucleases degrade nucleic acid polymers in cell free systems.

In some embodiments, the compound is combined with the sample at a temperature optimal for biological activity of the nucleic acids within the operating parameters of the compounds (usually between 5° C. and 50° C., with reduced stability of the compounds at higher temperatures). For in vitro assays, the compound can be combined with the sample at about room temperature (23° C.). At room temperature, detectable fluorescence in a solution of nucleic acids is essentially instantaneous depending on the sensitivity of the instrumentation that is used; fluorescence in solutions is generally visible by eye within 5 seconds after the compound is added, and is generally measurable within 2 to 5 minutes, although reaching equilibrium staining may take longer. Where a biological process is underway during in vitro analysis (e.g. in vitro transcription, replication, splicing, or recombination), the rapid labeling that occurs with the subject compounds avoids perturbation of biological system that is being observed. Gel staining at room temperature usually takes from 5 minutes to 2 hours depending on the thickness of the gel and the percentage of agarose or polyacrylamide, as well as the degree of cross-linking. In some embodiments, post-stained minigels stain to equilibrium in 20-30 minutes. For cells and other biological structures, transport of compounds across membranes is required whether the membranes are intact or disrupted. For preferred embodiments, visibly detectable fluorescence is obtained at room temperature within 15-20 minutes of incubation with cells, commonly within about 5 minutes. Some embodiments give detectable fluorescence inside cells in less than or equal to about 2 minutes. Lymphocytes loaded with 5 μM compound solutions give a fluorescence response in less than or equal to 5 seconds. This property is useful for observing nuclear structure and rearrangement, for example such as occurs during mitosis or apoptosis. Some of the compounds are generally not permeant to live cells with intact membranes; other compounds are generally permeant to eukaryotes but not to prokaryotes; still other compounds are only permeant to cells in which the cell membrane integrity has been disrupted (e.g. some dead cells). The relative permeability of the cell membrane to the compounds is determined empirically, e.g. by comparison with staining profiles or staining patterns of killed cells. The compound with the desired degree of permeability, and a high absorbance and quantum yield when bound to nucleic acids, is selected to be combined with the sample.

Fluorescence of the Compound-Nucleic Acid Complex.

The nucleic acid-compound complex formed during the staining or labeling of the sample with a compound of the present disclosure comprises a nucleic acid polymer non-covalently bound to one or more molecules of compound. The combination of compound and nucleic acid results in a fluorescent signal that is significantly enhanced over the fluorescence of the compound alone. In some embodiments, fluorescence of the compound-nucleic acid complex decreases at pH lower than 6.5 or greater than or equal to 8, but can be restored by returning to moderate pH.

Because the fluorescence for most of this class of compounds in solution is extremely low, the absolute degree of enhancement is difficult to determine. The quantum yield of unbound compound can be at or below 0.01, e.g., at or below 0.002, or at or below 0.001, which would yield a maximum enhancement of 100× or above, 500× or above, and 1000× or above, respectively. The level of fluorescence enhancement of the bound compound is generally many-fold greater than that of unbound compound (see Table 1 below), e.g., 900-1100 fold, or 1001-1100 fold for selected compounds, such that the compounds have a readily detectable increase in quantum yield upon binding to nucleic acids. The molar absorptivity (extinction coefficient) at the longest wavelength absorption peak of the compounds can be at or above 50,000, e.g., at or above 60,000 for the compounds where n=0; for compounds where n=1 or 2, the molar absorptivity can be greater than or equal to 90,000. Compounds with high extinction coefficients at the excitation wavelength are preferred for the highest sensitivity. A useful level of quantum yield in combination with other attributes of the subject compounds, including selectivity for rate of permeation, for binding affinity and/or the selectivity of excitation and emission bands to suit specific instrumentation, make the compounds useful for a very wide range of applications.

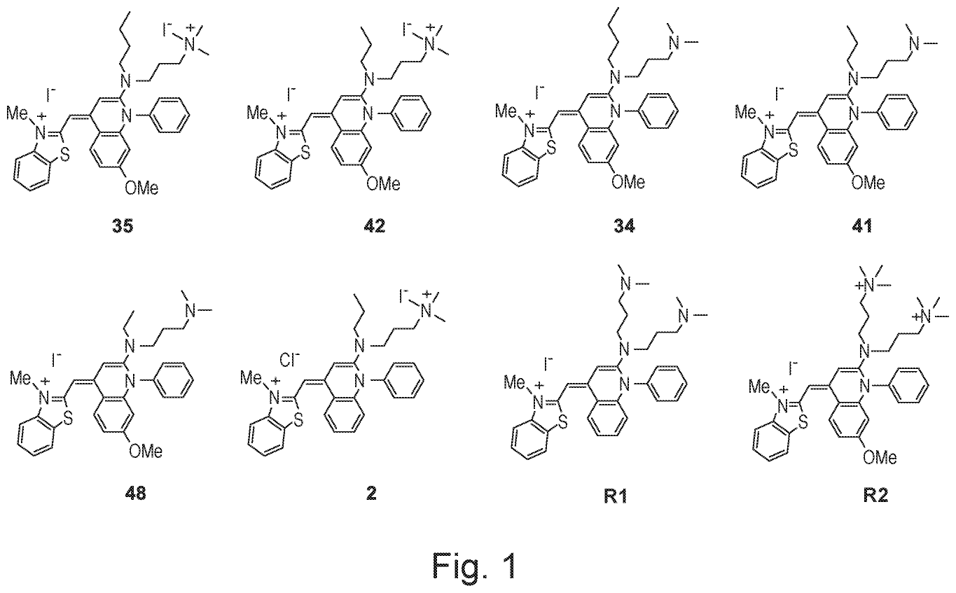

The presence, location, and distribution of nucleic acid are detected using the spectral properties of the fluorescent compound-nucleic acid complex. Spectral properties means any parameter that may be used to characterize the excitation or emission of the compound-nucleic acid complex including absorption and emission wavelengths, fluorescence polarization, fluorescence lifetime, fluorescence intensity, quantum yield, and fluorescence enhancement. In some embodiments, the spectral properties of excitation and emission wavelength are used to detect the compound-nucleic acid complex. The wavelengths of the excitation and emission bands of the compounds vary with compound composition to encompass a wide range of illumination and detection bands. This allows the selection of individual compounds for use with a specific excitation source or detection filter. In particular, complexes formed with compounds having a monomethine bridge (n=0) generally match their primary excitation band with the commonly used argon laser (488 nm) or HeCd laser (442 nm); whereas those with compounds with a trimethine bridge (n=1) primarily tend to match long wavelength excitation sources such as green HeNe (543 nm), the orange HeNe laser (594 nm), the red HeNe laser (633 nm), mercury arc (546 nm), or the Kr laser (568 or 647 nm); and complexes formed with compounds having a pentamethine bridge (n=2) primarily match very long excitation sources such as laser diodes or light emitting diodes (LEDs). In addition to the primary excitation peak in the visible range, the compound-nucleic acid complexes of the disclosure have a secondary absorption peak that permits excitation with UV illumination (FIG. 1). Compounds with n=1 and n=2 form complexes that permit excitation beyond 600 nm.

In some embodiments, the sample is excited by a light source capable of producing light at or near the wavelength of maximum absorption of the fluorescent complex, such as an ultraviolet or visible wavelength emission lamp, an arc lamp, a laser, or even sunlight or ordinary roomlight. In some embodiments the sample is excited with a wavelength within 20 nm of the maximum absorption of the fluorescent complex. Although excitation by a source more appropriate to the maximum absorption band of the nucleic acid-compound complex results in higher sensitivity, the equipment commonly available for excitation of samples can be used to excite the compounds of the present disclosure.

The fluorescence of the complex is detected qualitatively or quantitatively by detection of the resultant light emission at a wavelength of greater than or equal to about 450 nm, in some embodiments greater than or equal to about 480 nm, such as at greater than or equal to about 500 nm. Compounds having a quinolinium ring system usually absorb and emit at longer wavelength maxima than similarly substituted compounds having a pyridinium ring system. The emission is detected by means that include visual inspection, CCD cameras, video cameras, photographic film, or the use of current instrumentation such as laser scanning devices, fluorometers, photodiodes, quantum counters, plate readers, epifluorescence microscopes, scanning microscopes, confocal microscopes, flow cytometers, capillary electrophoresis detectors, or by means for amplifying the signal such as a photomultiplier tube. Many such instruments are capable of utilizing the fluorescent signal to sort and quantitate cells or quantitate the nucleic acids. Compounds can be selected to have emission bands that match commercially available filter sets such as that for fluorescein or for detecting multiple fluorophores with several excitation and emission bands.

Use of Complex.

Once the compound-nucleic acid complex is formed, its presence may be detected and used as an indicator of the presence, location, or type of nucleic acids in the sample, or as a basis for sorting cells, or as a key to characterizing the sample or cells in the sample. Such characterization may be enhanced by the use of additional reagents, including fluorescent reagents. The nucleic acid concentration in a sample can also be quantified by comparison with known relationships between the fluorescence of the nucleic acid-compound complex and concentration of nucleic acids in the sample.

In one aspect of the disclosure, the compound-nucleic acid complex is used as a means for detecting the presence or location of nucleic acids in a sample, where the sample is stained with the compound as described above, and the presence and location of a fluorescent signal indicates the presence of nucleic acids at the corresponding location. The fluorescent signal is detected by eye or by the instrumentation described above. The general presence or location of nucleic acids can be detected in a static liquid solution, or in a flowing stream such as a flow cytometer, or in a centrifugation gradient, or in a separation medium, such as a gel or electrophoretic fluid, or when leaving the separation medium, or affixed to a solid or semisolid support. Alternatively, the compound is selective for a particular type of nucleic acid, and the presence or location of particular nucleic acids are selectively detected.

Nucleic acid polymers can be detected with high sensitivity in a wide variety of solutions and separation media, including electrophoretic gels such as acrylamide and agarose gels, both denaturing and non-denaturing, and in other electrophoretic fluids or matrices, such as in capillary electrophoresis or micro-capillary electrophoresis. Compounds of the disclosure can give a strong fluorescent signal with small nucleic acid polymers (as few as 8 bases or base pairs with some embodiments) even with very small amounts of nucleic acids. In some embodiments, a single nucleic acid molecule can be detected, e.g., in a fluorescence microscope. Nucleic acid content from as few as 5 mammalian cells can be detected in cell extracts. As little as 100 picograms of dsDNA/mL of solution can be detected, e.g., in a fluorometer. In some embodiments, e.g. in conjunction with an ultraviolet transilluminator, it is possible to detect as little as 10 picograms of ds DNA per band in an electrophoretic gel; some compounds give such a bright signal even with illumination by ordinary fluorescent room lights, that as little as 1 ng DNA per band is detected.

Alternatively, the presence or location of nucleic acids, stained as above, can in turn be used to indicate the presence or location of organisms, cells, microvesicles, or organelles containing the nucleic acids, or the presence or location of nucleic acids in the cytosol or cytoplasm, or the presence or location of extracellular nucleic acids, where the presence or location of the fluorescent signal corresponds to the presence or location of the biological structure (e.g. stained cells or organelles) or free nucleic acid. Infective agents such as bacteria, mycoplasma, mycobacteria, viruses and parasitic microorganisms, as well as other cells, can be stained and detected inside of eukaryote cells, although the fluorescent signal generated by an individual virus particle is below the resolution level of standard detection instrumentation. In a further embodiment of the disclosure the fluorescent signal resulting from formation of the compound-nucleic acid complex is used as a basis for sorting cells, for example sorting stained cells from unstained cells or sorting cells with one set of spectral properties from cells with another set of spectral properties.

In addition to detection of the presence or location of nucleic acids as well as their enclosing structures, the staining profile that results from the formation of the compound-nucleic acid complex is indicative of one or more characteristics of the sample. By staining profile is meant the shape, location, distribution, spectral properties of the profile of fluorescent signals resulting from excitation of the fluorescent compound-nucleic acid complexes. The sample can be characterized simply by staining the sample and detecting the staining profile that is indicative of a characteristic of the sample. More effective characterization is achieved by utilizing a compound that is selective for a certain characteristic of the sample being evaluated or by utilizing an additional reagent (as described below), where the additional reagent is selective for the same characteristic to a greater or lesser extent or where the additional reagent is selective for a different characteristic of the same sample. The compounds of the disclosure can exhibit varying degrees of selectivity, e.g. with regard to nucleic acid structure, location, or cell type, or with regard to cell permeability.

In one embodiment of the disclosure, where the compound is selected to be membrane permeable or relatively impermeant to cell membranes, the staining profile that results from the formation of the compound-nucleic acid complex is indicative of the integrity of the cell membrane, which in turn is indicative of cell viability. The cells are stained as above for a time period and compound concentration sufficient to give a detectable fluorescent signal in cells with compromised membranes. The required time period is dependent on temperature and concentration, and can be optimized by standard procedures within the general parameters as previously described. Relatively impermeant compounds of the disclosure are used to indicate cells where the cell membranes are disrupted. Where the compound selected is impermeant to cells with intact membranes, formation of the fluorescent compound-nucleic acid complex inside the cell is indicative that the integrity of the cell membrane is disrupted and the lack of fluorescent compound-nucleic acid complexes inside the cell is indicative that the cell is intact or viable. The impermeant compound is optionally used in conjunction with a counterstain that gives a detectably different signal and is indicative of metabolically active cells or, in combination with the impermeant compound, is indicative of cells with intact membranes. Alternatively, the more permeant compounds of the disclosure are used to stain both cells with intact membranes and cells with disrupted membranes, which when used in conjunction with a counterstain that gives a detectably different signal in cells with disrupted membranes, allows the differentiation of viable cells from dead cells. The counterstain that gives a detectably different signal in cells with disrupted membranes is optionally an impermeant compound of the disclosure or another reagent that indicates loss of integrity of the cell membrane or lack of metabolic activity of the dead cells. When the cells are stained with a concentration of compound that is known to stain live bacteria, the relative reduction of fluorescence intensity can be used to distinguish quiescent bacteria, which are not actively expressing proteins, from metabolically active bacteria.

In a further embodiment of the disclosure, the shape and distribution of the staining profile of compound-nucleic acid complexes is indicative of the type of cell or biological structure that contains the stained nucleic acids. Cells may be discriminated by eye based on the visual fluorescent signal or be discriminated by instrumentation as described above, based on the spectral properties of the fluorescent signal. For example, compounds that are non-selective for staining nucleic acids in intracellular organelles can be used to identify cells that have an abundance or lack of such organelles, or the presence of micronuclei and other abnormal subparticles containing nucleic acids and characteristic of abnormal or diseased cells. A sample may be characterized as containing blebbing cells or nuclei based on the visible staining profile. Compounds that are selective for the nucleic acids in a particular organelle (e.g. in the nucleus or in mitochondria), even in the presence of limited staining of nucleic acids in the cytoplasm or other organelles, can be used to characterize cells as containing or lacking such organelles based on the intensity as well as the location of the signal, allowing the use of instrumentation to characterize the sample. In some embodiments, the staining profile used to characterize the sample is indicative of the presence, shape, or location of organelles or of cells, where the cells are located in a biological fluid, in a tissue, or in other cells.

Furthermore, the differential permeability of bacterial and higher eukaryotic cells to some compounds allows selective staining of live mammalian cells with little or no staining of live bacteria. A compound selected to be permeant to bacteria can be used in combination with a compound that is only permeant to eukaryotes to differentiate bacteria in the presence of eukaryotes. Dead bacteria with compromised membranes, such as those in the phagovacuoles of active macrophages or neutrophils, may be rendered permeable to the compounds that are otherwise only permeant to eukaryotes, as a result of toxic agents produced by the phagocytic cells.

In another embodiment of the disclosure, the staining profile results from the formation of the compound-nucleic acid complex in an electrophoretic gel, or sedimentation or centrifugation gradient. In addition to indicating the presence of nucleic acids in the gel, the staining profile is indicative of one or more characteristics of the nucleic acid solution applied to the gel. The number of bands and/or the intensity of the signal per band of the staining profile, for example, is indicative of the purity or homogeneity of the nucleic acid solution. Band tightness and degree of smearing is indicative of the integrity of the nucleic acid polymers in the solution. The size, conformation, and composition of the polymers, are indicated by the relative mobility of the polymer through the gel, which can be used to detect changes caused by interaction of analytes with the nucleic acid polymer such as protein binding or enzymatic activity. In some embodiments, the compounds have low intrinsic fluorescence so there is no need to destain gels to remove free compound. Furthermore, the fluorescence of the compound-nucleic acid complex is not quenched by denaturants such as urea and formaldehyde, eliminating the need for their removal from the gels prior to staining.

In yet another embodiment of the disclosure, the staining profile is indicative of the presence or predominance of a type of nucleic acid that is used to characterize the sample. In one embodiment of the disclosure, the compound is chosen to be more selective for AT or GC rich polymers, such that staining profile is indicative of the relative proportion of these bases. In another embodiment of the disclosure, the spectral properties of the nucleic acid-compound complex vary depending on the secondary structure of the nucleic acid present in the complex. In some embodiments, the spectral properties will vary in fluorescence enhancement, fluorescence polarization, fluorescence lifetime, excitation wavelength or emission wavelength. A comparison of the fluorescence response of a sample of unknown nucleic acids with that of a stained nucleic acid of known secondary structure allows the secondary structure of the unknown nucleic acids to be determined, and the amount of nucleic acids in the sample to be quantified. In this manner, RNA and single-stranded DNA can be differentiated from double-stranded DNA. Where nuclease is added to the nucleic acid polymers in solution or in fixed cells to digest the RNA or DNA prior to combining with the compound, the fluorescent signal from the compound-nucleic acid complex can be used to discriminate the nucleic acid polymer that was not digested in the presence of the nuclease from undigested polymers.

This same property of sensitivity to secondary structure by monomethine compounds can be used to quantitate ds nucleic acids in the presence of ss nucleic acids. Samples containing both ds and ss DNA or RNA can yield emission maxima in both the green and longer wavelength regions at high compound:base ratios. Meaningful information about the amounts of ss and ds nucleic acids in solution can be gathered by a direct comparison of the spectra of the low compound ratio sample and high compound ratio sample. For example, where a nucleic acid solution such as purified oligonucleotides, DNA amplification reactions, a cDNA synthesis, plasmid preparation, or cell extraction is stained with a high compound concentration (i.e. greater than or equal to the concentration of nucleic acid bases), the fluorescent signal that results from complexes formed by ss nucleic acids is red-shifted from the fluorescent signal formed by ds nucleic acids. Where the compound is selected to give a high quantum yield with ds nucleic acids and the quantum yield of the red-shifted fluorescent signal is minimal, the quantum yield of the stronger signal can be used to quantitate the amount of ds nucleic acid in the sample, even in the presence of ss nucleic acids.

The nucleic acids for this and other applications can be quantitated by comparison of the detectable fluorescent signal from the compound-nucleic acid complex, with a fluorescent standard characteristic of a given amount of nucleic acid. Where one type of nucleic acid in a sample is selectively digested to completion, the fluorescent signal can be used to quantitate the polymer remaining after digestion. Alternatively, prior to being stained, a solution of nucleic acid polymers is separated into discrete fractions using standard separation techniques and the amount of nucleic acid present in each fraction is quantitated using the intensity of the fluorescent signal that corresponds to that portion. The solution may be purified synthetic or natural nucleic acids or crude mixtures of cell extracts or tissue homogenates. Where aliquots from a single sample are taken over time, and the nucleic acid content of each aliquot is quantitated, the rate of cell or nucleic acid proliferation is readily determined from the change in the corresponding fluorescence over time.

In another aspect of the disclosure, the compound-nucleic acid complex is used as a fluorescent tracer or as probe for the presence of an analyte. In one aspect of the disclosure, the compound-nucleic acid complex is used as a size or mobility standard, such as in electrophoresis or flow cytometry. Alternatively, the fluorescent signal that results from the interaction of the compound with nucleic acid polymers can be used to detect or quantitate the activity or presence of other molecules that interact with nucleic acids. The nucleic acid polymers used to form the compound-nucleic acid complex are optionally attached to a solid or semi-solid support, such as described above, or is free in solution, or is enclosed in a biological structure. Such molecules include drugs, other compounds, proteins such as histones or ds or ss DNA or RNA binding proteins, or enzymes such as endonucleases or topoisomerases. In one aspect of the disclosure, a compound having a binding affinity for nucleic acid greater than that of the analyte being assayed displaces the analyte or prevents the interaction of the analyte with the nucleic acid polymer. For example, DNA templates that are heavily bound with a high affinity compound (e.g., at ratios of greater than or equal to about 3 bp:compound molecule in the staining solution) can be protected from DNase I activity. In some embodiments, the compounds having a binding affinity greater than or equal to 10⁻⁶ M, such as greater than or equal to 10⁻⁸ M, are effective to displace analytes that interact with nucleic acids. Compound affinity is determined by measuring the fluorescence of the compound-nucleic acid complex, fitting the resulting data to an equilibrium equation and solving for the association constant. In another aspect of the disclosure, compounds having a binding affinity that is less than that of the analyte being assayed are displaced from the compound-nucleic acid complex by the presence of the analyte, with the resultant loss of fluorescence. For example, lower affinity compound molecules prebound to double-stranded DNA are displaced by histones.

In one embodiment, the complex is used as an indicator of enzymatic activity, that is, as a substrate for nucleases, topoisomerases, gyrases, and other enzymes that interact with nucleic acids. Alternatively, the complex is used to quantitate the abundance of proteins (such as histones) that bind nucleic acids, or of DNA binding drugs (such as distamycin, spermine, actinomycin, mithramycin, chromomycin). The fluorescent complex is combined with the sample thought to contain the analyte and the resultant increase or decrease in fluorescent signal qualitatively or quantitatively indicates the presence of the analyte.

Additional Reagents.

The compounds of the disclosure can be used in conjunction with one or more additional reagents that are separately detectable. The additional reagents may be separately detectable if they are used separately, e.g. used to stain or label different aliquots of the same sample or if they stain or label different parts or components of a sample, regardless of whether the signal of the additional reagents is detectably different from the fluorescent signal of the compound-nucleic acid complex. Alternatively, the compound of the disclosure is selected to give a detectable response that is different from that of other reagents desired to be used in combination with the subject compounds. In some embodiments the additional reagent or reagents are fluorescent and have different spectral properties from those of the compound-nucleic acid complex. For example, compounds that form complexes that permit excitation beyond 600 nm can be used in combination with commonly used fluorescent antibodies such as those labelled with fluorescein isothiocyanate or phycoerythrin. Any fluorescence detection system (including visual inspection) can be used to detect differences in spectral properties between compounds, with differing levels of sensitivity. Such differences include, but are not limited to, a difference in excitation maxima, a difference in emission maxima, a difference in fluorescence lifetimes, a difference in fluorescence emission intensity at the same excitation wavelength or at a different wavelength, a difference in absorptivity, a difference in fluorescence polarization, a difference in fluorescence enhancement in combination with target materials, or combinations thereof. The detectably different compound is optionally one of the compounds of the disclosure having different spectral properties and different selectivity. In one aspect of the disclosure, the compound-nucleic acid complex and the additional detection reagents have the same or overlapping excitation spectra, but possess visibly different emission spectra, e.g., having emission maxima separated by ≥10 nm, ≥20 nm, or ≥50 nm. Simultaneous excitation of all fluorescent reagents may require excitation of the sample at a wavelength that is suboptimal for each reagent individually, but optimal for the combination of reagents. Alternatively, the additional reagent(s) can be simultaneously or sequentially excited at a wavelength that is different from that used to excite the subject compound-nucleic acid complex. In yet another alternative, one or more additional reagents are used to quench or partially quench the fluorescence of the compound-nucleic acid complex, such as by adding a second reagent to improve the selectivity for a particular nucleic acid or the AT/GC selectivity.

The additional compounds are optionally used to differentiate cells or cell-free samples containing nucleic acids according to size, shape, metabolic state, physiological condition, genotype, or other biological parameters or combinations thereof. The additional reagent is optionally selective for a particular characteristic of the sample for use in conjunction with a non-selective reagent for the same characteristic, or is selective for one characteristic of the sample for use in conjunction with a reagent that is selective for another characteristic of the sample. In one aspect of the disclosure, the additional compound or compounds are metabolized intracellularly to give a fluorescent product inside certain cells but not inside other cells, so that the fluorescence response of the cyanine compound of the disclosure predominates only where such metabolic process is not taking place. Alternatively, the additional compound or compounds are specific for some external component of the cell such as cell surface proteins or receptors, e.g. fluorescent lectins or antibodies. In yet another aspect of the disclosure, the additional compound or compounds actively or passively cross the cell membrane and are used to indicate the integrity or functioning of the cell membrane (e.g. calcein AM or BCECF AM). In another aspect, the additional reagents bind selectively to AT-rich nucleic acids and are used to indicate chromosome banding. In another aspect of the disclosure, the additional reagent is an organelle stain, i.e. a stain that is selective for a particular organelle, for example the additional reagent(s) may be selected for potential sensitive uptake into the mitochondria (e.g. rhodamine 123 or tetramethyl rosamine) or for uptake due to pH gradient in an organelle of a live cell (e.g. Diwu, et al., CYTOMETRY supp.7, p 77, Abstract 426B (1994)).

The additional compounds are added to the sample being analyzed to be present in an effective amount, with the optimal concentration of compound determined by standard procedures generally known in the art. Each compound is optionally prepared in a separate solution or combined in one solution, depending on the intended use. After illumination of the dyed cells at a suitable wavelength, as above, the cells are analyzed according to their fluorescence response to the illumination. In addition, the differential fluorescence response can be used as a basis for sorting the cells or nucleic acids for further analysis or experimentation. For example, all cells that “survive” a certain procedure are sorted, or all cells of a certain type in a sample are sorted. The cells can be sorted manually or using an automated technique such as flow cytometry, according to the procedures known in the art, such as in U.S. Pat. No. 4,665,024 to Mansour, et al. (1987).

Synthesis.

A useful synthetic route to the compounds of the present disclosure can be described in three parts, following the natural breakdown in the description of the compounds. In general, the synthesis of these compounds uses two or three precursors: a benzazolium salt, a methylpyridinium (or methylquinolinium) salt (both of which have the appropriate chemical substituents, or can be converted to the appropriate substituents), and (where n=1 or 2) a carbon source for the ethylene spacer(s). The chemistry that is used in the individual steps to prepare and combine these precursors so as to yield any of the subject compounds is generally well-understood by one skilled in the art. Although there are many possible variations that may yield an equivalent result, provided herein are general methods for their synthesis and incorporation of chemical modifications.

The Benzazolium Moiety.

A wide variety of derivatives of this type for use in preparing photographic compounds have been described, in particular by Brooker and his colleagues (Brooker, et al., J. AM. CHEM. SOC., 64, 199 (1942)).

If the heterocycle of the precursor contains an O, the precursor compound is a benzoxazolium; if it contains an S, it is a benzothiazolium; if it contains a Se, it is a benzoselenazolium; and if it contains a second N or alkyl substituted N, it is a benzimidazolium. The commercial availability of suitable starting materials and relative ease of synthesis make compounds containing O or S exemplary intermediates.

The benzazolium precursor will generally contain a substituent A on the carbon between the ring heteroatoms (N and O, S, Se, or a second N) whose nature is determined by the synthetic method utilized to couple the benzazolium precursor with the pyridinium or quinolinium precursor. When n=0, A is usually alkylthio, commonly methylthio, or A is chloro, bromo or iodo. For example, in 3-methyl-2-(methylthio)benzothiazolium tosylate, A is methylthio. When n=1 or 2, A is methyl. Only in the case of A=methyl is any part of A incorporated in the final compound.

The Quinolinium Moiety.

The strongly conjugated ring system of the compounds of the present disclosure allows resonance stabilization of the single positive charge on the ring atoms to be distributed over the entire molecule. In particular, the charge is stabilized by partial localization on each of the heterocyclic nitrogen atoms of the compound. As the subject compound is drawn herein (e.g., formula I shown above), the positive charge is formally localized on the benzazolium portion of the compound. However, it is commonly understood that a comparable resonance structure can be drawn in which the positive charge is formally localized on the quinolinium portion of the compound. Consequently, this latter portion of the molecule is generally referred to as a quinoline or quinolinium moiety, although in the resonance structure shown, it would formally be termed a dihydroquinoline.

Except where reference is to a specific pyridine or pyridinium salt, it is understood that mention of pyridines or pyridinium salts encompasses benzopyridines and benzopyridinium salts, which are formally called quinolines or quinolinium salts. Mention of quinolines and quinolinium salts refer only to structures containing two fused aromatic rings.

In the synthesis of the compounds of the disclosure, the second heterocyclic precursor is usually a quinolinium salt that is already appropriately substituted, e.g., at R¹. Alternatively, substituents can be incorporated into the quinolinium structure subsequent to attachment of the benzazolium portion of the compound.

In some embodiments, the quinolinium salt precursor contains a 6-membered pyridinium-based heterocycle in which a substituent B is located para to the ring nitrogen. When n=0, B is methyl, or B is chloro, bromo or iodo. When n=1 or 2, B is methyl.

There are several general methods for the synthesis of derivatives of pyridinium, including those derivatives having substituents at any available position, including substitutions that correspond to or can be converted to R¹, R⁵, and N(R²)(R³) of Formula (I). Such conversion can occur before or after reaction with the benzazolium portion to form the compound core structure.

Method 1. Alkylation of the nitrogen atom of an appropriately substituted quinoline with an alkylating agent such as a primary aliphatic halide, sulfate ester, sulfonate ester, epoxide or similar reagent directly yields a substituted quinolinium salt. For example, treatment of a quinoline with an alkyl iodide or dialkyl sulfate can be used to provide the corresponding alkyl substituent at R⁵.

Method 2. R⁵ substituents that are aryl or heteroaryl can be incorporated by an Ullmann reaction of aniline or a substituted aniline or of a pyridone or quinolone derivative. In this method, a diaryl amine or aryl-heteroaryl amine (generally commercially available) is condensed with diketene and acid to yield a 4-methyl-N-arylquinolone or a 4-methyl-N-heteroarylquinolone.

Quinolone intermediates containing a non-hydrogen group at R⁵ are useful as precursors to a wide variety of other pyridinium and quinolinium salts containing N(R²)(R³). E.g., a vinyl chloride salt is formed by treatment of the appropriate pyridone or quinolone with a strong chlorinating agent such as PCl₅, POCl₃ or SOCl₂. Similarly, a sulfonate can be substituted at R⁴ by treating the pyridone or quinolone with the appropriate sulfonic acid anhydride.

Halogen displacement. The reactivity of the 2-halogenated pyridinium or quinolinium intermediate allows for attachment of various N(R²)(R³) substituents. Of particular utility is the displacement of a 2-chloro substituent by amines. The displacement of chloride by amines is described in Example 1.

The methine bridge. The methine bridge consists of 1, 3 or 5 methine (—CH═) groups that bridge the benzazolium portion of the molecule and the pyridinium or quinolinium portion in such a way as to permit extensive electronic conjugation. The number of methine groups is determined by the specific synthetic reagents used in the synthesis.

When n=0, the synthesis of monomethine compounds commonly uses a combination of reagents where the methine carbon atom results from either A on the benzazolium salt or B on the pyridinium salt being methyl and the other of A or B being a reactive “leaving group” that such as methylthio or chloro, but which can be any leaving group that provides sufficient reactivity to complete the reaction. This type of reaction to make unsymmetrical monomethine compounds from two quaternary salts was originally described by Brooker et al., supra. Whether A or B is methyl depends primarily on the relative ease of synthesis of the requisite precursor salts. Because the compounds in this disclosure tend to vary on the pyridinium portion of the molecule; and furthermore, because 2-methyl and 4-methyl pyridines or quinolines are usually easier to prepare than their corresponding methylthio analogs, an exemplary choice is to prepare the subject monomethine compounds from precursors in which A=methylthio and B=methyl. The condensing reagent in the case of monomethine compounds can be a weak base such as triethylamine or diisopropylethylamine.

To synthesize trimethine compounds (n=1) both A and B are methyl. In this case the additional methine carbon is provided by a reagent such as diphenylforamidine, N-methylformanilide or ethyl orthoformate. Because under certain reaction conditions these same reagents can yield symmetrical cyanine compounds that incorporate two moles of a single quaternary salt, it is important to use the proper synthetic conditions, and a suitable ratio of the carbon-providing reactant to the first quaternary salt, so that the proper intermediate will be formed. This intermediate is treated either before or after purification with the second quaternary salt to form the asymmetric cyanine compound. If desired, the counterion Z⁻ can be exchanged at this point. Although one can usually react either of the heteroaromatic precursor salts with the carbon-providing reagent to form the required intermediate, an exemplary choice is to form the intermediate from the more readily available 2-methylbenzazolium salts as described by Brooker et al.

Synthesis of the pentamethine compounds (n=2) raises the same synthetic concerns about controlling the formation of an asymmetric intermediate. The three-carbon fragment that is required for the additional atoms in the bridge can be provided by a suitable precursor to malonaldehyde such as malonaldehyde dianil, 1,1,3,3-tetramethoxypropane, 1,1,3-trimethoxypropene, 3-(N-methylanilino)propenal or other reagents. The condensing agent for this reaction is usually 1-anilino-3-phenylimino-1-propene (U.S. Pat. No. 2,269,234 to Sprague, 1942), which generates the 2-(2-anilinovinyl)-3-methylbenzazolium tosylate intermediate.

The examples below are given so as to illustrate the practice of this disclosure. They are not intended to limit or define the entire scope of this disclosure.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1. Preparation of Compounds

Synthetic intermediates such as 1,2-dihydro-4-methyl-1-phenyl-2-quinolone (I1); 2-chloro-4-methyl-1-phenylquinolinium chloride (I2a); and 2-chloro-7-methoxy-4-methyl-1-phenylquinolinium chloride (I2b) are prepared as described in U.S. Pat. No. 5,658,751, which is incorporated by reference in its entirety.

The 4-methyl of such intermediates can be substituted, e.g., using 3-methyl-2-(methylthio)benzothiazolium tosylate to give a 4-[2,3-dihydro-3-methyl-(benzo-1,3-thiazol-2-yl)-methylidene] derivative. For example, the lithium enolate or silyl enolate of the quinoline is stirred with the benzothiazolium tosylate.

The 2-chloro of an intermediate such as (I2a) or (I2b), or a derivative thereof such as a 2-chloro-4-(2,3-dihydro-3-methyl(benzo-1,3-thiazol-2-yl)methylidene)-1-phenylquinolinium chloride prepared as described above, can be substituted by treatment with an amine, e.g., at 55° C. in 1,2-dichloroethane for about 1-2 hours. For example, N-(3-dimethylaminopropyl)-N-butylamine can be used to obtain compound 34, and N-(3-dimethylaminopropyl)-N-ethylamine can be used to obtain compound 48.

Ammoniumalkylamino-substituted compounds such as compounds 35 and 49 can be prepared from the corresponding tertiary amine (e.g., compound 34 or 48, respectively) by treatment with an excess of methyl iodide and PROTON-SPONGE (Aldrich) to methylate the dimethylamine and give the quaternary ammonium salt. Compounds 36, 37, 43, 44, 50, and 51 can be prepared analogously except that ethyl iodide or n-propyl iodide is used in place of methyl iodide.

Compounds such as 38, 45, and 52 containing a 2-hydroxyethyl group on the exocyclic quaternary amine can be prepared as above except that the corresponding hydroxyethyl iodide is used in place of the alkyl iodide. Compounds such as 39, 40, 46, 47, 53, and 54 can be prepared analogously, using an appropriately substituted benzyl iodide or bromide, e.g., benzyl iodide, benzyl bromide, or 3-methoxybenzyl bromide.

Routes for synthesis of other compounds disclosed herein can be determined by analogy to the above in view of known synthetic methods, including without limitation those described in U.S. Pat. No. 5,658,751.

### Example 2. Fluorescence Characteristics of the Disclosed Compounds

Compounds 1-65 were prepared and their fluorescence absorption and emission characteristics were measured. Intrinsic fluorescence of the compounds only (without DNA) was negligible. Emission spectra for the compounds bound to dsDNA were obtained by incubating 0.8 μM compound with 500 ng/ml calf thymus DNA in TE buffer (10 mM Tris-HCl, pH 7.5, 1 mM EDTA) in a final volume of 3 ml. Measurements were made using PerkinElmer LS 55 (F-11) Fluorescence spectrometer. Samples were excited at 488 nm and fluorescence emission was measured with emission slit widths of 2.5 nm. Absorption maxima were determined according to standard techniques.

Fluorescence enhancement measurements of the compounds were obtained by incubating 0.8 μM compound with 500 ng/ml calf thymus DNA with compounds at a final concentration of 0.8 μM in TE buffer. Compound 1 was used as an internal standard to control for variability (e.g., in excitation light intensity). The area-under-curve of fluorescence emission spectra from 500 nm to 715 nm was calculated, and compared to that of compound 1, which was defined as having 1000-fold fluorescence enhancement relative to fluorescence of the compound alone.

The UV/VIS absorption spectra were obtained with 10 μM compound solution in 50 mM potassium phosphate buffer (pH 7.0) in a final volume of 3 mL. Measurements were carried out using a Lambda 245 spectrophotometer from Perkin Elmer. Results are shown in Table 1.

TABLE 1
Fluorescence Characteristics

| | Absorbance | Emission | Fluorescence |
|---|---|---|---|
| Compound | Max (nm) | Max (nm) | Enhancement |
| 1 | 500 | 523 | 1000 |
| 2 | 501 | 523 | 964 |
| 3 | 501 | 524 | 997 |
| 4 | 501 | 524 | 925 |
| 5 | 501 | 523 | 954 |
| 6 | 502 | 523 | 979 |
| 7 | 502 | 522 | 978 |
| 8 | 495 | 525 | 946 |
| 9 | 496 | 525 | 908 |
| 10 | 496 | 526 | 926 |
| 11 | 496 | 524 | 903 |
| 12 | 496 | 525 | 937 |
| 13 | 496 | 523 | 922 |
| 14 | 497 | 526 | 907 |
| 15 | 495 | 524 | 864 |
| 16 | 496 | 523 | 863 |
| 17 | 496 | 523 | 865 |
| 18 | 496 | 524 | 882 |
| 19 | 497 | 524 | 884 |
| 20 | 497 | 523 | 871 |
| 21 | 491 | 522 | 639 |
| 22 | 497 | 520 | 639 |
| 23 | 498 | 524 | 622 |
| 24 | 497 | 522 | 630 |
| 25 | 497 | 523 | 640 |
| 26 | 498 | 521 | 653 |
| 27 | 498 | 521 | 668 |
| 28 | 504 | 525 | 672 |
| 29 | 506 | 528 | 648 |
| 30 | 506 | 526 | 638 |
| 31 | 506 | 527 | 642 |
| 32 | 507 | 527 | 672 |
| 33 | 507 | 526 | 652 |
| 34 | 488 | 517 | 1071 |
| 35 | 489 | 518 | 1069 |
| 36 | 489 | 521 | 1071 |
| 37 | 489 | 519 | 1089 |
| 38 | 489 | 520 | 1089 |
| 39 | 490 | 517 | 1080 |
| 40 | 490 | 520 | 1050 |
| 41 | 493 | 517 | 1020 |
| 42 | 494 | 519 | 1070 |
| 43 | 494 | 519 | 1033 |
| 44 | 494 | 518 | 1101 |
| 45 | 494 | 517 | 1093 |
| 46 | 494 | 520 | 931 |
| 47 | 495 | 518 | 1038 |
| 48 | 489 | 520 | 1016 |
| 49 | 490 | 520 | 1035 |
| 50 | 490 | 519 | 1020 |
| 51 | 490 | 520 | 1016 |
| 52 | 490 | 519 | 1031 |
| 53 | 490 | 520 | 1011 |
| 54 | 490 | 517 | 1008 |
| 55 | 496 | 520 | 908 |
| 56 | 499 | 523 | 894 |
| 57 | 496 | 521 | 877 |
| 58 | 496 | 521 | 874 |
| 59 | 498 | 520 | 863 |
| 60 | 500 | 522 | 878 |
| 61 | 514 | 540 | 26 |
| 62 | 519 | — | 0.3 |
| 63 | 518 | 543 | 42 |
| 64 | 527 | 529 | 204 |
| 65 | 527 | 530 | 136 |

### Example 3. In Vitro Selectivity

Six compounds (2, 34, 35, 41, 42, and 48) were characterized in in vitro selectivity experiments for double-stranded DNA (dsDNA) versus single-stranded DNA (ssDNA). These compounds were compared against comparative compounds R¹ and R². The structures of the tested compounds are shown in FIG. 1.

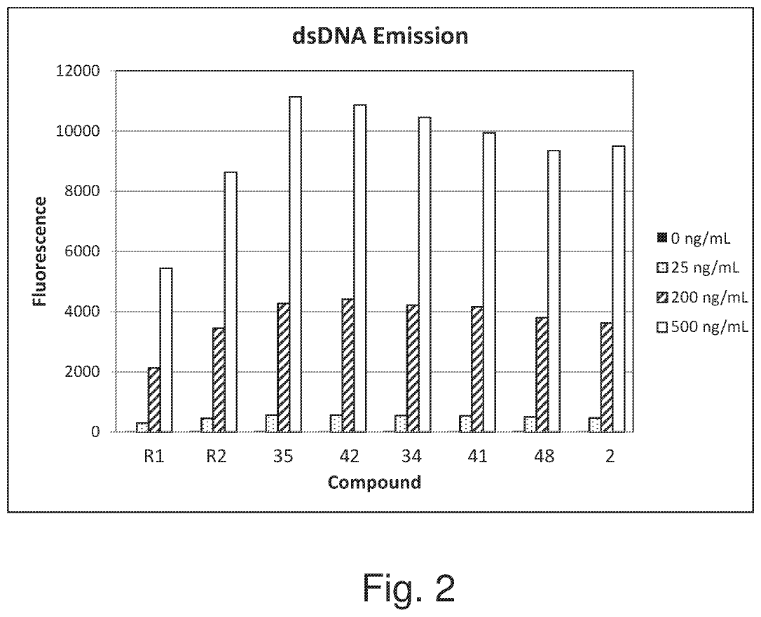

Compounds were tested in triplicate for fluorescence enhancement when bound to dsDNA. 190 μL of 1 μM compound in 1× Tris-EDTA, pH 7.5, 0.01% CHAPS was added to 10 μL of 4 different concentrations of dsDNA (0, 25 ng/mL, 200 ng/mL, and 500 ng/mL) in wells of 96 well Costar black, clear bottom microplates. Plates were read on a SpectraFluor multifunction microplate reader (Tecan) at 491 nm excitation and 520 nm emission. Fluorescence data with dsDNA are shown in FIG. 2 for the compounds.

Table 2 presents a comparison of fluorescent emission with 500 ng/mL dsDNA for different compounds in comparison to R1.

TABLE 2

| | Comparison of various compounds to R1 | | | | | | | |
|---|---|---|---|---|---|---|---|---|
| | for fluorescent emission with dsDNA | | | | | | | |
| | R1 | R2 | 35 | 42 | 34 | 41 | 48 | 2 |
| 500 | 5441 | 8627 | 11136 | 10860 | 10452 | 9936 | 9350 | 9494 |
| ng/mL | | | | | | | | |
| Ratio | 1.00 | 1.59 | 2.05 | 2.00 | 1.92 | 1.83 | 1.72 | 1.74 |
| to R1 | | | | | | | | |

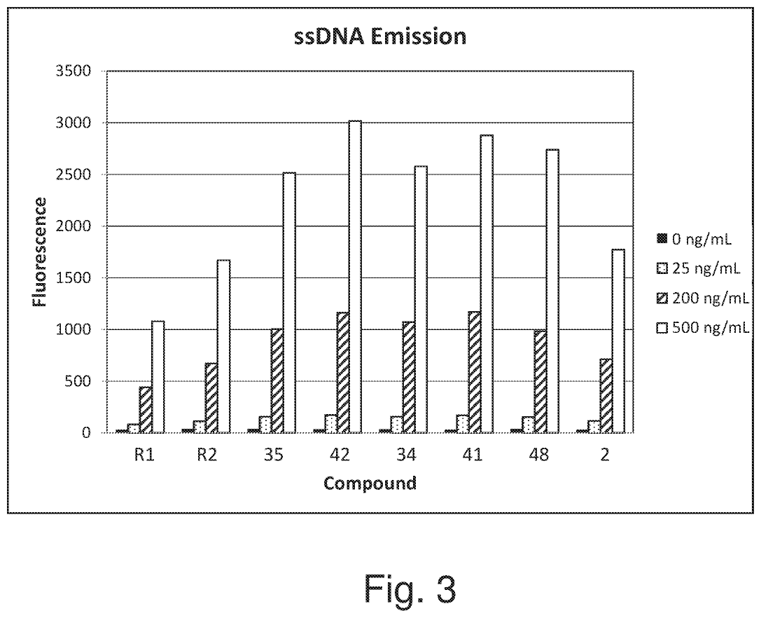

The compounds from FIG. 1 were also characterized with respect to their fluorescence enhancement when bound to ssDNA. The compounds from FIG. 1, R1 and R2 were tested in triplicate. 190 μL of 1 μM compound in 1× Tris-EDTA pH7.5, 0.01% CHAPS was added to 10 μL of 4 different concentrations of ssDNA in wells of 96 well Costar black, clear bottom microplates. Plates were read on a SpectraFluor multifunction microplate reader (Tecan) at 491 nm excitation and 520 nm emission. Fluorescence data with ssDNA are shown in FIG. 3 for the different tested compounds.

Table 3 presents a comparison of fluorescent emission at 0.5 ng/μL ssDNA for different compounds in comparison to R1.

TABLE 3

| | Comparison of various compounds to R1 | | | | | | | |
|---|---|---|---|---|---|---|---|---|
| | for fluorescent emission with ssDNA | | | | | | | |
| | R1 | R2 | 35 | 42 | 34 | 41 | 48 | 2 |
| 500 ng/mL | 1081 | 1669 | 2515 | 3017 | 2578 | 2878 | 2739 | 1773 |
| Ratio to R1 | 1.0 | 1.5 | 2.3 | 2.8 | 2.4 | 2.7 | 2.5 | 1.6 |

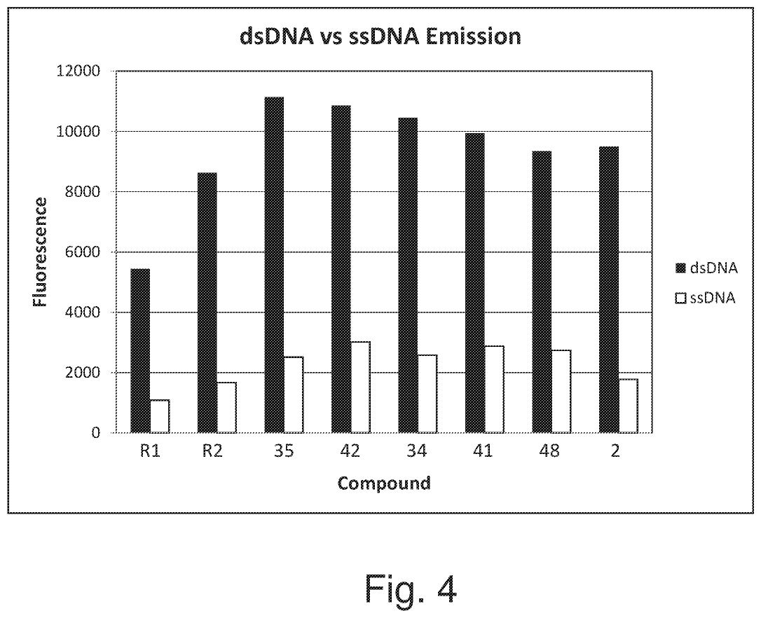

For each individual compound, the dsDNA and ssDNA fluorescence emission at 10 ng/μL was compared to evaluate in vitro selectivity for dsDNA/ssDNA, as shown in FIG. 4.

Table 4 presents the ratio of dsDNA emission versus ssDNA emission for the various compounds.

TABLE 4

| | Comparison of various compounds for | | | | | | | |
|---|---|---|---|---|---|---|---|---|
| | dsDNA and ssDNA emission | | | | | | | |
| | R1 | R2 | 35 | 42 | 34 | 41 | 48 | 2 |
| dsDNA | 5441 | 8627 | 11136 | 10860 | 10452 | 9936 | 9350 | 9494 |
| ssDNA | 1081 | 1669 | 2515 | 3017 | 2578 | 2878 | 2739 | 1773 |
| ratio | 5.0 | 5.2 | 4.4 | 3.6 | 4.1 | 3.5 | 3.4 | 5.4 |

### Example 4. Further In Vitro Selectivity Experiments

Further in vitro selectivity experiments for double-stranded DNA (dsDNA) versus single-stranded DNA (ssDNA) were performed using the methods outlined in Example 3 to compare compounds 2, 34, 41, 42, and 48 to comparative compounds R1 and R2.

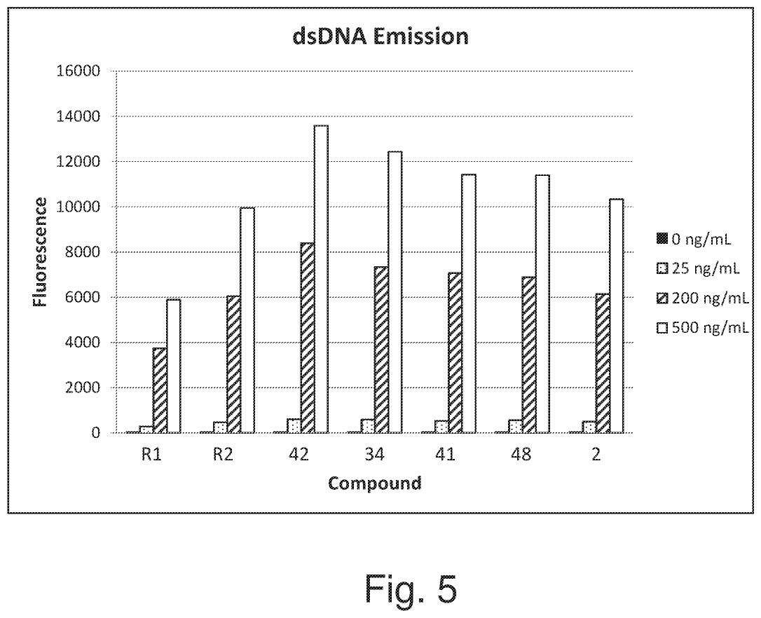

Compounds were tested for fluorescence enhancement when bound to dsDNA as described in Example 3. Fluorescence data with dsDNA are shown in FIG. 5 for the different tested compounds.

Table 5 presents a comparison of fluorescent emission with 0.5 ng/μL dsDNA for different compounds in comparison to R1.

TABLE 5

| | Comparison of various compounds to R1 for | | | | | | |
|---|---|---|---|---|---|---|---|
| | fluorescent emission with dsDNA | | | | | | |
| | R1 | R2 | 42 | 34 | 41 | 48 | 2 |
| 500 ng/mL | 5887 | 9941 | 13580 | 12434 | 11424 | 11400 | 10339 |
| Ratio to R1 | 1.0 | 1.7 | 2.3 | 2.1 | 1.9 | 1.9 | 1.8 |

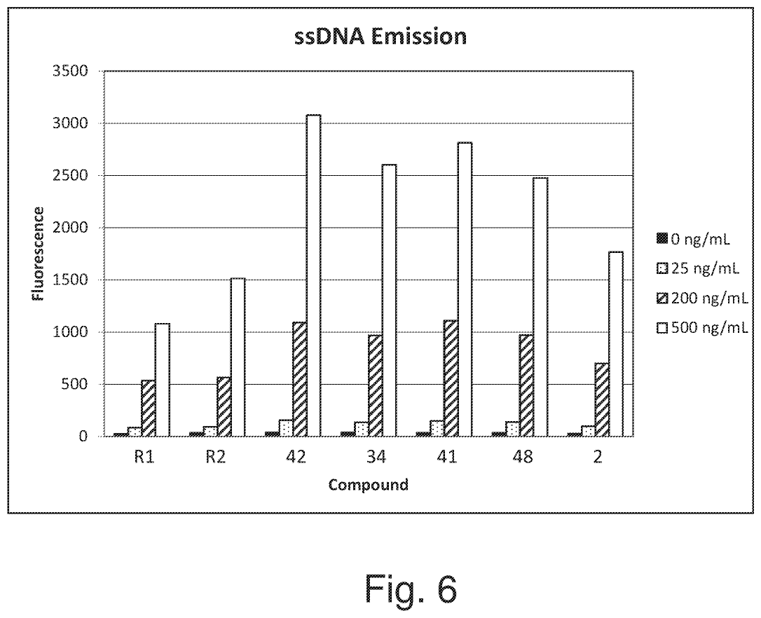

Next, the compounds were compared for their fluorescence enhancement when bound to ssDNA as described in Example 1. Fluorescence data with ssDNA are shown in FIG. 6 for the different tested compounds.

Table 6 presents a comparison of fluorescent emission at 500 ng/mL ssDNA for different compounds in comparison to R2.

TABLE 6

| | Comparison of various compounds to R2 for | | | | | | |
|---|---|---|---|---|---|---|---|
| | fluorescent emission with ssDNA | | | | | | |
| | R1 | R2 | 42 | 34 | 41 | 48 | 2 |
| 500 ng/mL | 1080 | 1512 | 3078 | 2603 | 2814 | 2477 | 1767 |
| Ratio to R2 | 0.7 | 1.0 | 2.9 | 2.4 | 2.6 | 2.3 | 1.6 |

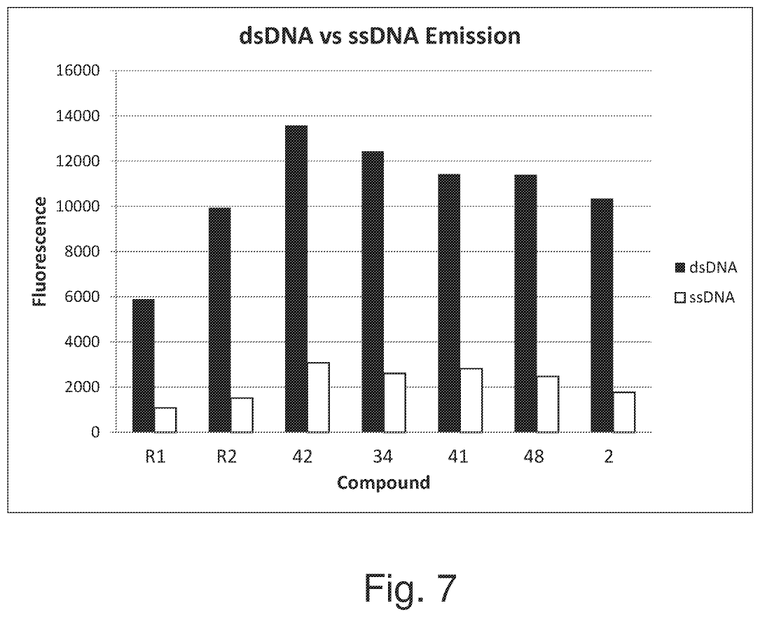

For each individual compound, the dsDNA and ssDNA fluorescence emission at 10 ng/μL was calculated to estimate in vitro selectivity, as shown in FIG. 7.

Table 7 presents the ratio of dsDNA emission versus ssDNA emission for the various compounds.

TABLE 7

| | Comparison of various compounds for | | | | | | |
|---|---|---|---|---|---|---|---|
| | dsDNA and ssDNA emission | | | | | | |
| | R1 | R2 | 42 | 34 | 41 | 48 | 2 |
| dsDNA | 5887 | 9941 | 13580 | 12434 | 11424 | 11400 | 10339 |
| ssDNA | 1080 | 1512 | 3078 | 2603 | 2814 | 2477 | 1767 |
| ratio | 5.5 | 6.6 | 4.4 | 4.8 | 4.1 | 4.6 | 5.9 |

### Example 5. Evaluation of New Compounds by Fluorescence Microscopy

The ability of different compounds to label cytosolic and nuclear DNA in confocal microscopy is analyzed as described below.

Experimental. The mouse prostate tumor cell line TRAMP-C2, which had been treated with Plasmocin (Invivogen) to exclude potential mycoplasma contaminations, is cultured in DMEM medium (Invitrogen) supplemented with 10% FCS (Hyclone), 50 μM 2-mercaptoethanol, 200 μM asparagine, 2 mM glutamine (Sigma) and 1% pen/strep (Invitrogen). Cells are stained with 3 l/ml compound for 90 min at 37° C. after fixation with 4% paraformaldehyde. Labelled cells are analyzed using a confocal scanning microscope equipped with a 100× oil immersion objective and an ApoTome optical sectioning device (Zeiss). Mean fluorescence intensity (MFI) values in a given field of view are normalized to the signal obtained using comparative compound R¹ as the control fluorescent DNA stain; normalized MFI values range from 2% to 690%.

What is claimed is:

1. A compound of formula (I): 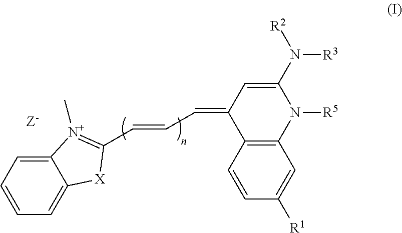
wherein:
Z⁻ is a biologically acceptable counterion;
X is S, O, Se, or NR^{N1}, where R^{N1} is H or C₁₋₆ alkyl;
n is 0, 1, or 2;
R¹ is H or —O—C₁₋₆ alkyl;
R² is C₂₋₆ alkyl;
R³ is —(CH₂)ₘNRR′, or —(CH₂)ₘN⁺RR′R″, wherein:
m is 2-6,
R and R′ are each independently a substituted or unsubstituted aryl or heteroaryl, or a substituted or unsubstituted C₁₋₆ alkyl, and
R″ is H, a substituted or unsubstituted aryl or heteroaryl, or a substituted or unsubstituted C₁₋₆ alkyl; and
R⁵ is an alkyl, alkenyl, polyalkenyl, alkynyl or polyalkynyl group having 1-6 carbons;
a substituted or unsubstituted aryl or heteroaryl; or a substituted or unsubstituted cycloalkyl having 3-10 carbons;
wherein if R³ is —(CH₂)₃—N(CH₃)₂ or —(CH₂)₃—N⁺(CH₃)₃, then R² is ethyl or C₄₋₆ alkyl.

2. The compound of claim 1, wherein R¹ is —O—C₁₋₄ alkyl.

3. The compound of claim 2, wherein R¹ is —O-methyl.

4. The compound of any one of the preceding claims, wherein R² is C₁₋₄ alkyl.

5. The compound of any one of claims 1 to 4, wherein R² is ethyl or C₄₋₆ alkyl.

6. The compound of claim 5, wherein R² is ethyl.

7. The compound of claim 5, wherein R² is n-propyl.

8. The compound of claim 5, wherein R² is n-butyl.

9. The compound of any one of the preceding claims, wherein R³ is —(CH₂)₃—N(CH₃)₂.

10. The compound of any one of claims 1 to 8, wherein R³ is —(CH₂)ₘN⁺RR′R″.

11. The compound of claim 10, wherein R is C₁₋₆ alkyl and R′ and R″ are each methyl.

12. The compound of claim 11, wherein R is ethyl.

13. The compound of claim 11, wherein R is n-propyl.

14. The compound of any one of claims 10 to 13, further comprising a biologically acceptable counterion Zₐ⁻ associated with R³.

15. The compound of claim 14, wherein Zₐ⁻ is a halide, sulfate, an alkanesulfonate, an arylsulfonate, phosphate, perchlorate, tetrafluoroborate, tetraarylboride, nitrate, or an anion of an aromatic or aliphatic carboxylic acid.

16. The compound of claim 15, wherein Zₐ⁻ is chloride, bromide, iodide, an alkanesulfonate, an arylsulfonate, or perchlorate.

17. The compound of claim 16, wherein Zₐ⁻ is bromide.

18. The compound of claim 16, wherein Zₐ⁻ is iodide.

19. The compound of claim 16, wherein Zₐ⁻ is chloride.

20. The compound of any one of the preceding claims, wherein R⁵ is a substituted or unsubstituted aryl or heteroaryl; or a substituted or unsubstituted cycloalkyl having 3-10 carbons.

21. The compound of claim 20, wherein R⁵ is a substituted or unsubstituted aryl or heteroaryl.

22. The compound of claim 21, wherein R⁵ is unsubstituted phenyl or phenyl substituted with 1, 2, or 3 instances of C₁₋₄ alkyl.

23. The compound of claim 21, wherein R⁵ is unsubstituted phenyl.

24. The compound of any one of the preceding claims, wherein Z⁻ is a halide, sulfate, an alkanesulfonate, an arylsulfonate, phosphate, perchlorate, tetrafluoroborate, tetraarylboride, nitrate, or an anion of an aromatic or aliphatic carboxylic acid.

25. The compound of claim 23, wherein Z⁻ is chloride, bromide, iodide, an alkanesulfonate, an arylsulfonate, or perchlorate.

26. The compound of claim 24, wherein Z⁻ is bromide.

27. The compound of claim 24, wherein Z⁻ is iodide.

28. The compound of claim 24, wherein Z⁻ is chloride.

29. The compound of any one of the preceding claims, wherein X is S.

30. The compound of any one of the preceding claims, wherein n is 0.

31. The compound of any one of the preceding claims, which is a compound of formula (II):

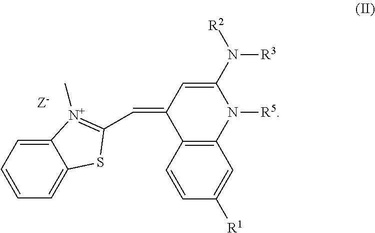

32. The compound of claim 30, which is a compound of formula (III):

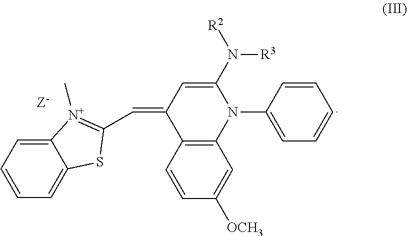

33. The compound of claim 31 or 32, wherein:
R² is C₂₋₆ alkyl; and
R³ is —(CH₂)ₘNRR′, or —(CH₂)ₘN⁺RR′R″, wherein m is 2-6, and R, R′ and R″ are each independently a substituted or unsubstituted aryl or heteroaryl, or a substituted or unsubstituted C₁₋₆ alkyl;
wherein if R³ is —(CH₂)₃—N(CH₃)₂ or —(CH₂)₃—N⁺(CH₃)₃, then R² is ethyl or C₄₋₆ alkyl.

34. The compound of claim 33, wherein R² is ethyl or n-butyl.

35. The compound of claim 31 or 32, wherein:
R² is C₂₋₆ alkyl;
R³ is —(CH₂)ₘN⁺RR′R″;
R is C₁₋₄ alkyl which is unsubstituted or substituted with hydroxyl, or aryl which is unsubstituted or substituted with methyl, ethyl, or —O—CH₃; and
R′ and R″ are each independently C₁₋₆ alkyl.

36. The compound of any one of claims 31, 32, or 35, wherein R² is ethyl, n-propyl, or n-butyl.

37. The compound of any one of claims 31, 32, 35, or 36, wherein:
R³ is —(CH₂)ₘN⁺RR′R″;
R is ethyl or n-propyl; and
R′ and R″ are each methyl.

38. The compound of any one of claims 31, 32, 35, or 36, wherein:
R³ is —(CH₂)ₘN⁺RR′R″;
R is —CH₂CH₂OH; and
R′ and R″ are each methyl.

39. The compound of any one of claims 31, 32, 35, or 36, wherein:
R³ is —(CH₂)ₘN⁺RR′R″;
R is phenyl which is unsubstituted or substituted with methyl, ethyl, or —O—CH₃; and
R′ and R″ are each methyl.

40. The compound of claim 39, wherein R is phenyl or 3-methoxyphenyl.

41. The compound of claim 1, which is:

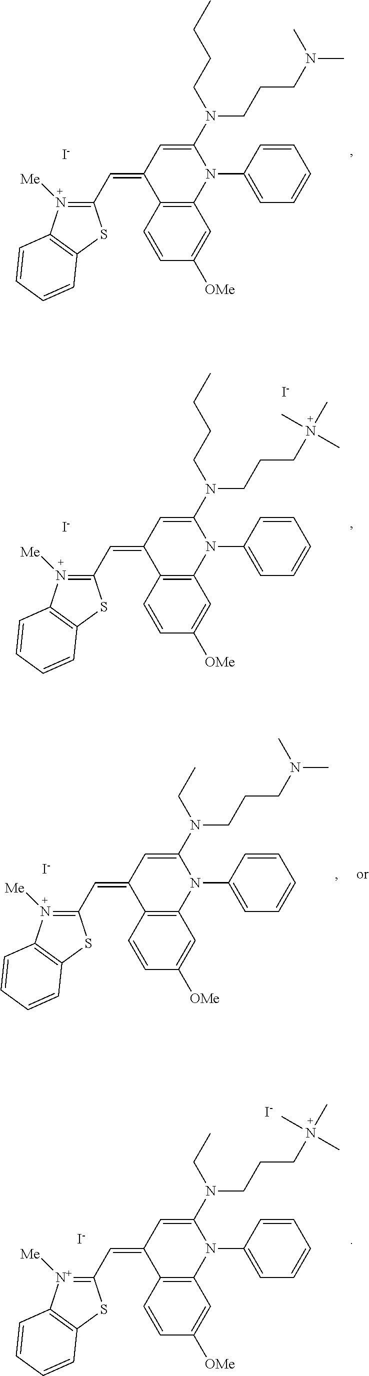

42. The compound of claim 1, which is:

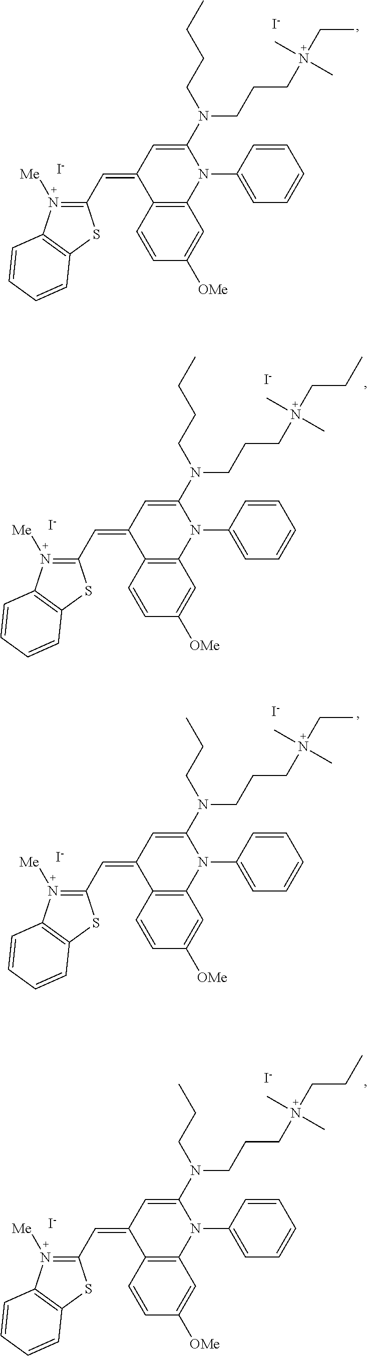

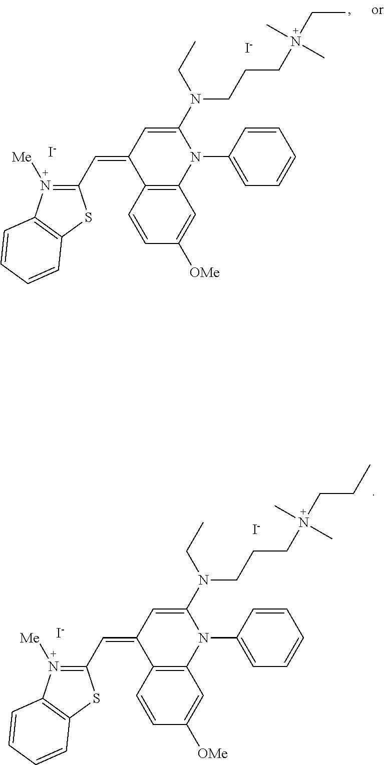

43. The compound of claim 1, which is:

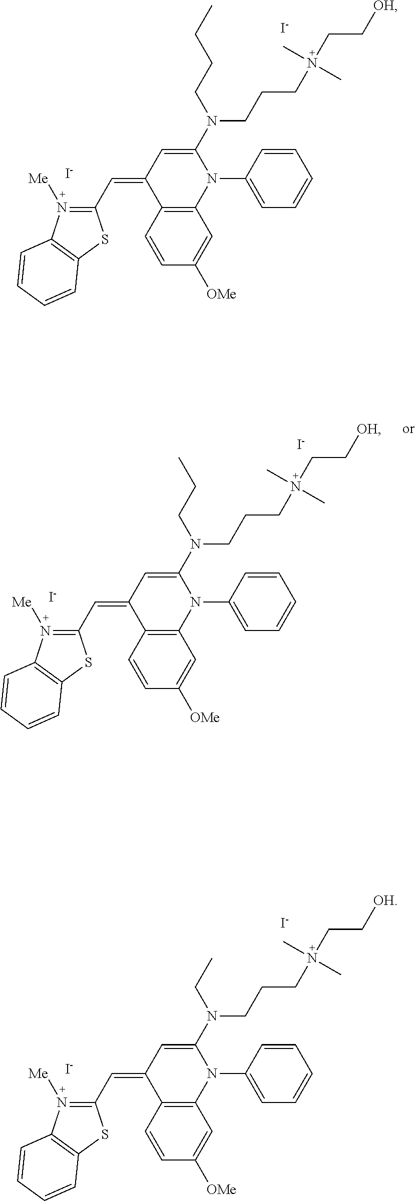

44. The compound of claim 1, which is:

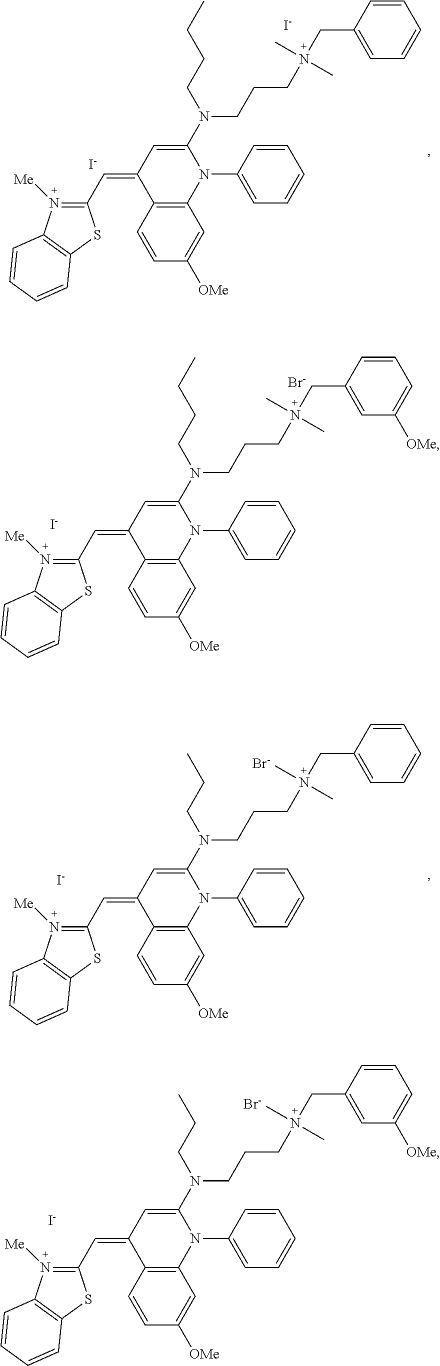

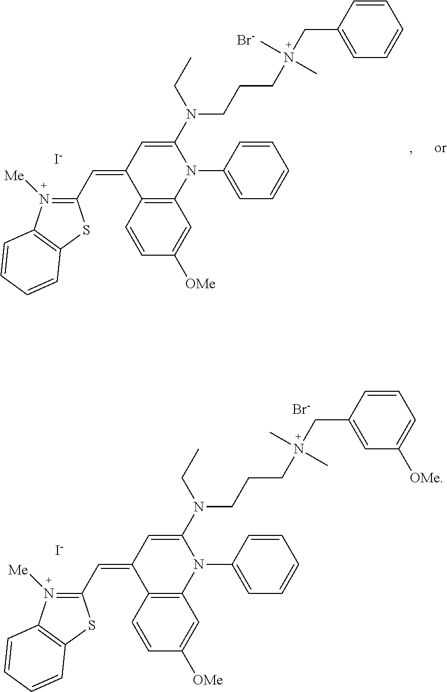

45. A method of staining a nucleic acid comprising contacting the nucleic acid with a compound according to any one of claims 1 to 44.

46. A method of labeling a nucleic acid comprising contacting the nucleic acid with a compound according to any one of claims 1 to 44.

47. A fluorescent complex comprising a compound according to any one of claims 1 to 44 non-covalently associated with a nucleic acid.

48. A fluorescent complex formed by the method of claim 45 or 46.

49. The method of claim 45 or 46 or the fluorescent complex of claim 47 or 48, wherein the nucleic acid is dsDNA.

50. The method of claim 45 or 46 or the fluorescent complex of claim 47 or 48, wherein the nucleic acid is ssDNA.

51. The method of claim 45 or 46 or the fluorescent complex of claim 47 or 48, wherein the nucleic acid is RNA or an RNA-DNA hybrid.

52. The method of claim 45 or 46 or the fluorescent complex of any one of claims 47 to 51, wherein the nucleic acid has a length of about 8 to about 15 nucleotides, about 15 to about 30 nucleotides, about 30 to about 50 nucleotides, about 50 to about 200 nucleotides, about 200 to about 1000 nucleotides, about 1 kb to about 5 kb, about 5 kb to about 10 kb, about 10 kb to about 50 kb, about 50 kb to about 500 kb, about 500 kb to about 5 Mb, about 5 Mb to about 50 Mb, or about 50 Mb to about 500 Mb.

53. The method of claim 45 or 46 or the fluorescent complex of any one of claims 47 to 51, wherein the nucleic acid is a plasmid, cosmid, PCR product, restriction fragment, or cDNA.

54. The method of claim 45 or 46 or the fluorescent complex of any one of claims 47 to 51, wherein the nucleic acid is genomic DNA.

55. The method of claim 45 or 46 or the fluorescent complex of any one of claims 47 to 51, wherein the nucleic acid is a natural or synthetic oligonucleotide.

56. The method or fluorescent complex of any one of claims 45 to 55, wherein the nucleic acid comprises modified nucleic acid bases or links.

57. The method or fluorescent complex of any one of claims 45 to 55, wherein the nucleic acid is in an electrophoresis fluid or matrix.

58. The method or fluorescent complex of any one of claims 45 to 55, wherein the nucleic acid is in a cell.

59. The method or fluorescent complex of any one of claims 45 to 55, wherein the nucleic acid is in an organelle, virus, viroid, cytosol, cytoplasm, or biological fluid.

60. The method or fluorescent complex of any one of claims 45 to 55, wherein the nucleic acid is in or was obtained from a water sample, soil sample, foodstuff, fermentation process, or surface wash.

61. A method of detecting a nucleic acid comprising exciting the fluorescent complex of any one of claims 47 to 60 and detecting fluorescently emitted light.

62. A method of detecting a nucleic acid in a sample, the method comprising:
a) combining a compound according to any one of claims 1 to 44 with a sample that contains or is thought to contain a nucleic acid;
b) incubating the sample and the compound for a sufficient amount of time for the compound to combine with the nucleic acid in the sample to form a compound-nucleic acid complex;
c) illuminating the compound-nucleic acid complex with an appropriate wavelength to form an illuminated mixture; and
d) detecting fluorescently emitted light thereby detecting the nucleic acid present in the illuminated mixture.

63. The method of claim 61 or 62, wherein exciting the fluorescent complex comprises exposing the fluorescent complex to light with a wavelength ranging from about 460 nm to about 520 nm, about 470 nm to about 510 nm, about 480 nm to about 510 nm, about 485 nm to about 505 nm, or about 490 nm to about 495 nm.

64. The method of any one of claims 61 to 63, wherein the fluorescently emitted light is detected with a microscope, plate reader, fluorimeter, or photomultiplier tube.

65. The method of any one of claims 61 to 64, further comprising quantifying the nucleic acid.

66. A method of detecting a biological structure, the method comprising:
a) combining a sample that contains or is thought to contain a specific biological structure with a compound of any one of claims 1 to 44;
b) incubating the combined sample and compound for a time sufficient for the compound to combine with nucleic acids in the biological structure to form a pattern of compound-nucleic acid complexes having a detectable fluorescent signal that corresponds to the biological structure; and
c) detecting the fluorescent signal that corresponds to the biological structure.

67. The method of claim 66, wherein the biological structure is a prokaryotic cell, a eukaryotic cell, a virus or a viroid.

68. The method of claim 66, wherein the biological structure is a subcellular organelle that is intracellular or extracellular.

69. A method of determining cell membrane integrity, the method comprising:
a) incubating a sample containing one or more cells with a compound according to any one of claims 1 to 44 for a time sufficient for the compound to combine with intracellular nucleic acids to form an intracellular compound-nucleic acid complex having a detectable fluorescent signal; and
b) determining cell membrane integrity of the one or more cells based on presence of the detectable fluorescent signal, where the presence of the detectable fluorescent signal indicates that the cell membrane integrity is compromised and the absence of the detectable fluorescent signal indicates that the cell membrane integrity is intact.

70. A method of quantitating nucleic acids in a sample, the method comprising:
a) combining a compound according to any one of claims 1 to 44 with a sample that contains or is thought to contain a nucleic acid;
b) incubating the sample and the compound for a sufficient amount of time for the compound to combine with nucleic acid in the sample to form a compound-nucleic acid complex;
c) illuminating the compound-nucleic acid complex with an appropriate wavelength to form an illuminated mixture; and
d) quantifying the nucleic acid present in the illuminated mixture based on comparison of the detectable fluorescent signal in the illuminated mixture with a fluorescent standard characteristic of a given amount of a nucleic acid.

71. A kit for detecting nucleic acid in a sample, wherein the kit comprises a compound according to any one of claims 1 to 44 and an organic solvent.

72. The kit of claim 71, further comprising instructions for detecting nucleic acid in a sample.

73. A staining solution comprising a compound according to any one of claims 1 to 44 and a detergent or an organic solvent.
